(19) United States
(12) Patent Application Publication
(10) Pub. No.: US 2021/0244365 A1
(43) Pub. Date: Aug. 12, 2021

(54) NON-INVASIVE EPIDERMAL HEALTH-MONITORING SENSOR, PATCH SYSTEM AND METHOD, AND EPIDEMIOLOGICAL MONITORING AND TRACKING SYSTEM RELATED THERETO

(71) Applicant: SPECTRONIX INC., Gatineau (CA)

(72) Inventor: Patrick Assouad, Ottawa (CA)

(21) Appl. No.: 17/231,991

(22) Filed: Apr. 15, 2021

Related U.S. Application Data
(63) Continuation-in-part of application No. 17/046,704, filed on Oct. 9, 2020.
(60) Provisional application No. 62/670,291, filed on May 11, 2018.
(60) Provisional application No. 63/012,147, filed on Apr. 18, 2020.
(60) Provisional application No. 63/105,223, filed on Oct. 24, 2020.
(60) Provisional application No. 63/125,367, filed on Dec. 14, 2020.

(51) Int. Cl. A61B 5/00; A61B 5/1455; A61B 5/145; A61B 5/01; A61B 5/11; G16H 40/67; G16H 50/30

(52) U.S. Cl. CPC A61B 5/7275; A61B 5/14552; A61B 5/14546; A61B 5/6833; G16H 50/30; A61B 5/6814; A61B 5/1112; A61B 5/0022; G16H 40/67; A61B 5/01

(57) ABSTRACT

Described are various embodiments of non-invasive epidermal health-monitoring sensor, patch, system and method, and epidemiological monitoring and tracking system related thereto.

### CROSS REFERENCE TO RELATED APPLICATIONS

This application is a continuation-in-part of U.S. patent application Ser. No. 17/046,704 filed Oct. 9, 2020 as a national phase entry of International Application No. PCT/CA2019/050637 filed May 10, 2019, which claims priority to U.S. Provisional Application No. 62/670,291 filed May 11, 2018. This application also claims priority to U.S. Provisional Application No. 63/012,147 filed Apr. 18, 2020, U.S. Provisional Application No. 63/105,223 filed Oct. 24, 2020 and U.S. Provisional Application No. 63/125,367 filed Dec. 14, 2020, the entire disclosure of each of which is hereby incorporated herein by reference.

### FIELD OF THE DISCLOSURE

The present disclosure relates to health-monitoring systems, and, in particular, to a non-invasive epidermal health-monitoring sensor, patch, system and method, and epidemiological monitoring and tracking system related thereto.

### BACKGROUND

Hospital-grade health monitoring equipment encompasses a gamut of sensors and monitoring systems designed to specifically probe and address various health markers in different medical context, such as surgical, emergency and intensive care units. A critically ill patient may be fitted with a standard pulse oximeter clip on their finger to monitor variations in blood oxygen saturation, and electrocardiograph probes for cardiac monitoring and related vital signs. Regular body temperature readings may also be taken by medical staff using handheld, and recently contactless, thermometers.

In some recent developments, as reported by Chung et al. in the article “Binodal, wireless epidermal electronic systems with in-sensor analytics for neonatal intensive care”, Science 363, 947 (2019), the entire contents of which are hereby incorporated herein by reference, respective ECG/temperature and PPG/temperature epidermal sensors have been proposed for neonatal intensive care units.

While such equipment and practices may become commonplace and practical in a hospital or critical care setting, their general cost, size in standard bedside equipment, and ofttimes complex operation make them inadequate for residential or widespread use amongst non-medical staff.

Accordingly, in the context of home treatment or monitoring, for instance, when addressing residential patients or again dealing with imposed or recommended public self-isolation, quarantine, or like considerations as applicable in a local or regional outbreak or epidemic, or outright pandemic, such equipment becomes unreasonable, leaving individuals empty-handed to track their conditions beyond basic symptom progression tracking and use of standard household devices like a handheld thermometer.

Furthermore, efforts for the mass collection and compilation of home care, national or even global medical health care data for statistical analysis or epidemiological tracking are generally challenged by inaccurate or inconsistent data collection at best, leading to potentially questionable, incomplete or inaccurate results.

This background information is provided to reveal information believed by the applicant to be of possible relevance. No admission is necessarily intended, nor should be construed, that any of the preceding information constitutes prior art or forms part of the general common knowledge in the relevant art.

### SUMMARY

The following presents a simplified summary of the general inventive concept(s) described herein to provide a basic understanding of some aspects of the disclosure. This summary is not an extensive overview of the disclosure. It is not intended to restrict key or critical elements of embodiments of the disclosure or to delineate their scope beyond that which is explicitly or implicitly described by the following description and claims.

A need exists for a health-monitoring sensor, system and method, such as a non-invasive epidermal health-monitoring sensor, patch, system and method, and/or an epidemiological monitoring and tracking system related thereto, that overcome some of the drawbacks of known techniques, or at least, provides a useful alternative thereto. In particular, some aspects of the herein described embodiments provide a relatively affordable and non-medical user-friendly device and/or system to track health-related variables in a non-medical context, for example, at home, in a residence, in self-isolation or under prescribed or recommended social distancing environments, in large-scale quarantine centers or the like.

A need also or alternatively exists for a broad spectrum oximetry device, system and method that overcome some of the drawbacks of known oximetry solutions, or at least, provide a useful alternative thereto. Examples of such systems, devices and methods are disclosed herein, in accordance with difference embodiments.

In accordance with some aspects, there is provided a patient health monitoring system comprising: an epidermal device to be affixed to the patient's skin and comprising: an optical spectroscopy probe operable to acquire data representative of blood oxygenation levels; and a wireless interface; a mobile application operable on a mobile device to interface with said wireless interface and receive therefrom said acquired data; stored computer-executable code operable by a digital processor to monitor for variations in said blood oxygenation levels and digitally evaluate said variations against preset variations corresponding to benchmark blood oxygenation profiles, wherein said profiles are digitally associated with a preset blood oxygenation index defining at least a lower health risk rating and a higher health risk rating, and output a signal representative of said higher health risk rating in response to said evaluation.

In one embodiment, the blood oxygenation levels comprise deoxyhemoglobin concentrations, and wherein said benchmark blood oxygenation profiles comprises deoxyhemoglobin concentration profiles.

In one embodiment, blood oxygenation levels comprise respective deoxyhemoglobin concentrations and oxidized hemoglobin concentrations, and wherein said benchmark blood oxygenation profiles comprise dissolved oxygen profiles derived from said concentrations.

In one embodiment, blood oxygenation levels comprise both arterial and venous blood oxygenation levels.

In one embodiment, the stored computer-executable code is stored on said mobile device and operable by said mobile application.

In one embodiment, the stored computer-executable code is stored on a server communicatively accessible by said mobile application via said mobile device.

In one embodiment, the system further comprises a remote server operatively linked to said mobile application to process acquired data from multiple users in continuously or periodically optimizing said benchmark profiles accordingly.

In one embodiment, the system further comprises a remote server operatively linked to said mobile application to process acquired data from multiple users in continuously or periodically updating a global health-related tracking.

In one embodiment, the epidermal device comprises an integrated epidermal patch further comprising a body temperature sensor.

In one embodiment, the system for monitoring a user supported by an oxygen-providing apparatus.

In one embodiment, the system for monitoring a user exposed to partial oxygen pressures deviating from a standard value of about 0.21 atm at Standard Temperature and Pressure (STP).

In one embodiment, the epidermal device comprises a cerebral device to be affixed to the user's head.

In one embodiment, the optical spectroscopy probe comprises a broad-spectrum oximetry probe comprising: a broad-spectrum light source providing broad-spectrum illumination to said user body region in probing multiple blood-related chromophores exhibiting distinguishable spectral responses; and a spectrometer operable to acquire an optical signal from said user body region resulting from said broad-spectrum illumination so to digitally capture said distinguishable spectral responses, wherein said blood-related chromophores are representative of said blood oxygenation levels; wherein said stored computer-executable code is operable by said digital processor to spectrally resolve said distinguishable spectral responses from said optical signal to isolate a spectral signature for a designated chromophore; and compare said isolated spectral signature with a designated set of corresponding signatures associated with a discriminable health-related condition; and output said signal representative of said higher health risk rating in response to said evaluation of said health-related indicator representative of said discriminable health-related condition.

In one embodiment, the digital processor is operable to extract an absolute concentration for said designated chromophore from said spectral signature.

In one embodiment, the broad-spectrum light source emits light in a range of about 600 nm to about 1000 nm.

In one embodiment, the spectrometer is operable to isolate respective spectral responses within at least 10 spectral regions within said broad-spectrum illumination.

In one embodiment, the chromophores comprise at least three of carbon monoxide, melanin, cytochrome oxidase, oxyhemoglobin, or deoxyhemoglobin.

In one embodiment, the discriminable health-related condition comprises at least one of: blood or tissue oxygenation, pulse, blood pressure, blood flow rate, blood loss or hemorrhaging, onset of blackouts or change in cognition, lung efficiency, rate of oxygen consumption by an organ of interest, psychological or physiological stress, presence of stroke, or a change in vital signs.

In one embodiment, the digital processor is operable to isolate a combined spectral signature for a designated combination of chromophores; and compare said isolated combined spectral signature with a designated set of corresponding combined signatures associated with said discriminable health-related condition.

In accordance with another aspect, there is provided a geographical health monitoring system comprising: a centralized health-monitoring server; a set of wearable health-monitoring devices to be affixed to respective users within a geographical area to: acquire health-related data from each of said respective users over time; concurrently track a location of each of said respective users; and communicate information related t said health-related data and said location to said centralized health-monitoring server for tracking; wherein, for each of said respective users, said health-related data is digitally compared with a designated health-related profile associated with a designated medical condition to automatically output a health risk indicator for a given location upon given health-related data acquired at said given location substantially aligning with said designated health-related profile.

In one embodiment, a geographical outbreak is automatically identified upon a group of said health risk indicators are output for a given area around a same said given location.

In one embodiment, infection transmissions are automatically tracked by tracking a geographical evolution of said health risk indicators over time.

In one embodiment, the geographical tracking of asymptomatic users is automatically implemented and retroactively evaluated by a digital processor upon any of said asymptomatic users later triggering a said health risk indicator so to track potential retroactive geographical infection transmission from said asymptomatic users.

In one embodiment, the designated health-related profile comprises a combination of at least two of a designated body temperature threshold, a blood oxygen-concentration related threshold or profile, a respiration rate or variation profile, a cardiac rate or variation profile, or a blood pressure or variation profile.

In one embodiment, each of said a set of wearable health-monitoring devices comprises an epidermal device to be affixed to the patient's skin and comprising: an optical spectroscopy probe operable to acquire data representative of blood oxygenation levels; wherein the system further comprises stored computer-executable code operable by a digital processor to monitor for variations in said blood oxygenation levels and digitally evaluate said variations against preset variations corresponding to benchmark blood oxygenation profiles, wherein said profiles are digitally associated with a preset blood oxygenation index defining at least a lower health risk rating and a higher health risk rating associated with said designated medical condition.

In one embodiment, the optical spectroscopy probe comprises a broad-spectrum oximetry probe comprising: a broad-spectrum light source providing broad-spectrum illumination to said user body region in probing multiple blood-related chromophores exhibiting distinguishable spectral responses; and a spectrometer operable to acquire an optical signal from said user body region resulting from said broad-spectrum illumination so to digitally capture said distinguishable spectral responses, wherein said blood-related chromophores are representative of said blood oxygenation levels; wherein said stored computer-executable code is operable by said digital processor to spectrally resolve said distinguishable spectral responses from said optical signal to isolate a spectral signature for a designated chromophore; and compare said isolated spectral signature with a designated set of corresponding signatures associated with said designated medical condition.

In accordance with another aspect, there is provided an oximetry system for monitoring a health-related condition in a user, the system comprising: a broad-spectrum oximetry probe fixable to a user body region, the probe comprising: a broad-spectrum light source providing broad-spectrum illumination to said user body region in probing multiple blood-related chromophores exhibiting distinguishable spectral responses; and a spectrometer operable to acquire an optical signal from said user body region resulting from said broad-spectrum illumination so to digitally capture said distinguishable spectral responses; a digital data processor operatively connected to said broad-spectrum oximetry probe, and programmed to: spectrally resolve said distinguishable spectral responses from said optical signal to isolate a spectral signature for a designated chromophore; and compare said isolated spectral signature with a designated set of corresponding signatures associated with a discriminable health-related condition; and output a health-related indicator representative of said discriminable health-related condition.

In one embodiment, the digital processor is operable to extract an absolute concentration for said designated chromophore from said spectral signature.

In one embodiment, the digital processor is operable to extract a variation in said spectral signature over time and compare said variation with said set of corresponding signatures to output said health-related indicator.

In one embodiment, the broad-spectrum light source comprises a full spectrum infrared (IR) light source.

In one embodiment, the broad-spectrum light source emits light in a range of about 600 nm to about 1000 nm.

In one embodiment, the spectrometer is operable to isolate respective spectral responses within at least 10 spectral regions within said broad-spectrum illumination.

In one embodiment, the spectrometer is operable to isolate respective spectral responses within at least 40 spectral regions within said broad-spectrum illumination.

In one embodiment, the spectrometer is operable to isolate respective spectral responses within at least 80 spectral regions within said broad-spectrum illumination.

In one embodiment, the user body region comprises a cerebral region.

In one embodiment, the broad-spectrum oximetry probe is integrated within a headband.

In one embodiment, the chromophores comprise at least three of carbon monoxide, melanin, cytochrome oxidase, oxyhemoglobin, or deoxyhemoglobin.

In one embodiment, the discriminable health-related condition comprises at least one of: blood or tissue oxygenation, pulse, blood pressure, blood flow rate, blood loss or hemorrhaging, onset of blackouts or change in cognition, lung efficiency, rate of oxygen consumption by an organ of interest, psychological or physiological stress, presence of stroke, or a change in vital signs.

In one embodiment, the digital processor is operable to isolate a combined spectral signature for a designated combination of chromophores; and compare said isolated combined spectral signature with a designated set of corresponding combined signatures associated with said discriminable health-related condition.

In accordance with another aspect, there is provided an oximeter for monitoring a health-related condition, the oximeter comprising: a broad-spectrum light source for providing broad-spectrum illumination to a user body region in probing multiple blood-related chromophores exhibiting distinguishable spectral responses; and a spectrometer operable to acquire an optical signal from said user body region resulting from said broad-spectrum illumination so to digitally capture said distinguishable spectral responses.

In one embodiment, the broad-spectrum light source comprises a broadband infrared (IR) light source.

In one embodiment, the broadband light source emits light in a range of about 600 nm to about 1000 nm.

In one embodiment, the spectrometer is operable to isolate respective spectral responses within at least 10 spectral regions within said broadband illumination.

In one embodiment, the spectrometer is operable to isolate respective spectral responses within at least 40 spectral regions within said broadband illumination.

In one embodiment, the spectrometer is operable to isolate respective spectral responses within at least 80 spectral regions within said broadband illumination.

In one embodiment, the oximeter is integrated within a headband.

In accordance with another aspect, there is provided a non-transitory computer-readable medium comprising digital instructions to be implemented by one or more digital processors to monitor one or more health-related parameters in a user, by: activating a broadband light source providing broadband illumination to a user body region in probing multiple blood-related chromophores exhibiting distinguishable spectral responses; acquiring, via a spectrometer, an optical signal from said user body region resulting from said broadband illumination so to digitally capture said distinguishable spectral responses; spectrally resolving said distinguishable spectral responses from said optical signal to isolate a spectral signature for a designated chromophore; comparing said isolated spectral signature with a designated set of corresponding signatures associated with a discriminable health-related condition; and outputting a health-related indicator representative of said discriminable health-related condition.

In one embodiment, the spectrally resolving comprises resolving respective optical signals within at least 10 distinct wavelength regions.

In one embodiment, the spectrally resolving comprises resolving respective optical signals within at least 40 distinct wavelength regions.

In one embodiment, the spectrally resolving comprises resolving respective optical signals within at least 80 distinct wavelength regions.

In one embodiment, the instructions are operable to extract an absolute concentration for said designated chromophore from said spectral signature.

In one embodiment, the instructions are operable to extract a variation in said spectral signature over time and compare said variation with said set of corresponding signatures to output said health-related indicator.

Other aspects, features and/or advantages will become more apparent upon reading of the following non-restrictive description of specific embodiments thereof, given by way of example only with reference to the accompanying drawings.

### BRIEF DESCRIPTION OF THE FIGURES

Several embodiments of the present disclosure will be provided, by way of examples only, with reference to the appended drawings, wherein:

FIG. 1 is a schematic diagram of a non-invasive epidermal health-monitoring sensor and system, in accordance with one embodiment.

FIG. 2 is a diagram of a monitoring method using the system of FIG. 1, in accordance with one embodiment.

FIG. 3 is an exemplary plot of the change in time of the relative absorbance of deoxyhemoglobin as measured by NIRS of an individual breathing a series of different gas mixtures containing lower than normal concentrations of oxygen (hypoxic mix), in accordance with one embodiment.

FIG. 4 is an exemplary plot of the change in time of the relative absorbance of deoxyhemoglobin as measured by NIRS of an individual breathing a series of different gas mixtures containing higher than normal concentrations of oxygen (hyperoxic mix), in accordance with one embodiment.

FIG. 5 is an exemplary plot of the change in time of the relative absorbance of both oxyhemoglobin and deoxyhemoglobin as measured by NIRS of an individual breathing normal air, an hyperoxic mix and normal air again, while changing position from a sitting position, a supine position and a sitting position again, in accordance with one embodiment;

FIG. 6 is an exemplary plot of the change in time of the relative molar concentration of cerebral deoxyhemoglobin as measured by NIRS of an individual breathing different gas mixtures and immersed in water at different depths, in accordance with one embodiment;

FIG. 7 is an exemplary plot of the relative change in concentration of cerebral deoxyhemoglobin as measured by NIRS of an individual breathing different gas mixtures inside a hyperbaric chamber, in accordance with one embodiment;

FIG. 8 is an exemplary plot of the relative change in time of the concentration of cerebral deoxyhemoglobin as measured by NIRS of an individual both changing positions (sitting or supine) and breathing different gas mixtures inside a hyperbaric chamber, in accordance with one embodiment;

FIG. 9 shows three exemplary plots illustrating the relative change over time in the concentration of cerebral deoxyhemoglobin; the heart rate and the breathing or respiration rate, from top to bottom respectively, of a user engaging in an underwater physical activity as a function of time, in accordance with one embodiment;

FIG. 10 shows a diagram of another method for monitoring a user's health risk for a user using an oxygen providing apparatus and/or inside a sealed pressurized environment, in accordance with one embodiment;

FIG. 11 is a schematic diagram of a broad or full spectrum oximetry (spectroximetry) system, in accordance with one embodiment;

FIG. 12 is a schematic diagram of an exemplary software processing system, in accordance with one embodiment;

FIG. 13 is a process flow diagram illustrating a monitoring method for assessing certain physiological parameters using the system of FIG. 1, in accordance with some embodiments;

FIG. 14 is an exemplary plot of a spectral variation measured in a user breathing normal air while sitting in a chair, in accordance with one embodiment;

FIG. 15 is an exemplary plot of an average change in recorded spectra when switching from normal air (21% O2) to a hypoxic gas containing 5% O2, in accordance with one embodiment;

FIG. 16 is an exemplary plot of an average change in recorded spectra when switching from normal air (21% O₂) to breathing pure oxygen (100% O₂), in accordance with one embodiment; and

FIG. 17 is a diagram of an epidemiological monitoring and tracking system operable at least in part, in one embodiment, in concert with or as part of the health monitoring system of FIG. 1.

Elements in the several figures are illustrated for simplicity and clarity and have not necessarily been drawn to scale. For example, the dimensions of some of the elements in the figures may be emphasized relative to other elements for facilitating understanding of the various presently disclosed embodiments. Also, common, but well-understood elements that are useful or necessary in commercially feasible embodiments are often not depicted in order to facilitate a less obstructed view of these various embodiments of the present disclosure.

### DETAILED DESCRIPTION

Various implementations and aspects of the specification will be described with reference to details discussed below. The following description and drawings are illustrative of the specification and are not to be construed as limiting the specification. Numerous specific details are described to provide a thorough understanding of various implementations of the present specification. However, in certain instances, well-known or conventional details are not described in order to provide a concise discussion of implementations of the present specification.

Various apparatuses and processes will be described below to provide examples of implementations of the system disclosed herein. No implementation described below limits any claimed implementation and any claimed implementations may cover processes or apparatuses that differ from those described below. The claimed implementations are not limited to apparatuses or processes having all of the features of any one apparatus or process described below or to features common to multiple or all of the apparatuses or processes described below. It is possible that an apparatus or process described below is not an implementation of any claimed subject matter.

Furthermore, numerous specific details are set forth in order to provide a thorough understanding of the implementations described herein. However, it will be understood by those skilled in the relevant arts that the implementations described herein may be practiced without these specific details. In other instances, well-known methods, procedures and components have not been described in detail so as not to obscure the implementations described herein.

In this specification, elements may be described as “configured to” perform one or more functions or “configured for” such functions. In general, an element that is configured to perform or configured for performing a function is enabled to perform the function, or is suitable for performing the function, or is adapted to perform the function, or is operable to perform the function, or is otherwise capable of performing the function.

It is understood that for the purpose of this specification, language of “at least one of X, Y, and Z” and “one or more of X, Y and Z” may be construed as X only, Y only, Z only, or any combination of two or more items X, Y, and Z (e.g., XYZ, XY, YZ, ZZ, and the like). Similar logic may be applied for two or more items in any occurrence of “at least one . . . ” and “one or more . . . ” language.

Unless defined otherwise, all technical and scientific terms used herein have the same meaning as commonly understood by one of ordinary skill in the art to which this invention belongs.

Throughout the specification and claims, the following terms take the meanings explicitly associated herein, unless the context clearly dictates otherwise. The phrase “in one of the embodiments” or “in at least one of the various embodiments” as used herein does not necessarily refer to the same embodiment, though it may. Furthermore, the phrase “in another embodiment” or “in some embodiments” as used herein does not necessarily refer to a different embodiment, although it may. Thus, as described below, various embodiments may be readily combined, without departing from the scope or spirit of the innovations disclosed herein.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or element(s) as appropriate.

The devices, systems and methods described herein provide, in accordance with different embodiments, different examples of a health-monitoring sensor, system and method, such as a a non-invasive epidermal health-monitoring sensor, patch, system and method In particular, some aspects of the herein described embodiments provide a relatively affordable and non-medical user-friendly device and/or system to track health-related variables in a non-medical context, for example, at home, in a residence, in self-isolation or under prescribed or recommended social distancing environments, in large-scale quarantine centers or the like.

For instance, embodiments as described herein include an integrated sensor that can be operated from skin contact on or near the patient or user's head (e.g. on the skin of the forehead), or in other body skin locations amenable to suitable body temperature and spectroscopic probing, that can not only improve upon applicable manufacturing and commercial implementation costs and user friendliness, but also yield improved health monitoring accuracy and latency. For instance, an integrated spectroscopic and body temperature skin patch, or like form factor, may provide for a compact solution to health monitoring in concurrently, and from an integrated and non-invasive form factor, monitor critical health-related parameters such as, but not limited to, body temperature (e.g. for the onset, escalation, critical threshold and/or drop of a fever), standard arterial blood oxygen saturation levels (e.g. arterial oxygen saturation monitoring for risk of hypoxia), enhanced blood oxygen indicators (e.g. arterial and/or venous deoxyhemoglobin concentration/variations, oxygenated hemoglobin concentrations/variations, derived dissolved, stored or discharged blood oxygen levels, hyperoxia risk monitoring, etc.), spectroscopic cardiac monitoring (e.g. heart rate, rhythm and/or pattern), spectroscopic respiratory system monitoring (e.g. respiratory rate, rhythm and/or pattern), blood pressure derived from spectroscopic readings (or obtained via a complementary sensor), etc.

Furthermore, in some embodiments, a cerebral integrated spectroscopic and body temperature solution is provided that can enhance, as compared to other skin patch locations, measures for body temperature (e.g. as compared to a predominantly arterial neck patch solution, for example), and reduce detection latency, and/or increase detectability and/or accuracy for critical blood-oxygen-related health risks/indicators. For instance, a standard pulse oximeter will clip to a patient's finger and thus capture less reliable blood saturation data therefrom. However, if the patient is cold, suffers from poor blood circulation, has a low pulse, suffers from a respiratory condition (which typically reduced blood circulation to extremities), or like conditions, standard pulse oximetry data acquired at body extremities may be of limited quality or lead to false or delayed results. For instance, in critical care context, the delay between gas exchange in the lungs and detection of changes in saturation at extremities has been shown to be clinically significant and reducing or eliminating this delay, for instance by operating a cerebral spectroscopy sensor as described herein, can result in improved patient outcomes.

Conversely, the implementation of cerebral blood spectroscopy via an integrated head or forehead-mounted patch or like sensor, can result in improved results as it relates to reliability, accuracy and latency. Namely, while blood flow may be reduced or constrained from body extremities for various reasons, cerebral blood flow will typically take precedence in most physiological systems and medical contexts, thus providing critical information in a more reliable manner. Furthermore, as variations in cerebral blood flow, oxygen saturation, arterial and/or venous oxidized hemoglobin and/or deoxyhemoglobin concentration, dissolved oxygen variations or related metrics can represent or expedite detection of the onset of critical health conditions (e.g. cerebral hyperoxia, hypoxia and/or global respiratory conditions), the implementation of a cerebral health-monitoring device or system may provide enhanced health monitoring and alerting capabilities. A forehead patch also provides a convenient form factor in that it will typically not interfere with habitual user activities, and thus, may be less likely to be removed, dislodged or tampered with.

As will be described in greater detail below, the integrated skin-contact health-monitoring device or system may be operable to communicatively link, for example, an integrated instrumented forehead skin patch with a wirelessly enabled mobile or portable device for local health tracking and/or data transmission to a remote health-monitoring server, database, center or clinic, for example. Accordingly, integrated health-monitoring data can be wirelessly, or less conveniently so, via wired connection, relayed to a local or remote user and/or monitoring interface for monitoring, historical tracking, trend tracking, population modelling and/or forecasting, medical intervention and/or planning (e.g. at the local, regional, provincial, state, federal, national, international and/or global level), alerting, and/or further processing. For example, while traditional health monitoring devices may be configured to track a single or limited health-related parameters, an integrated health-related sensor and system as described herein may be configured to not only locally track health-related parameters locally for the given user (both for respective but also combined comparative health-related data monitoring/alerting), but also leverage historical average, time-variable, or community or group-based evolution profiles of respective, combined or comparative health-related data acquisitions so provide greater screening, testing, or diagnostic capabilities and/or result in more accurate or predictively effective treatments, therapies and/or accommodations.

Furthermore, by combining vital sign monitoring as described herein, with communications and self-localization (e.g. GPS, cellular triangulation and/or other self-localization technologies, emergency contacts and/or services can be automatically alerted or contacted in the event of an emergency where the patient/user is otherwise unable to make direct contact (e.g. loss of consciousness, stroke, heart attack, hypoxia, etc.). In that respect, integration with self-localization technology, whether it be directly implemented within or in concert with the epidermal sensor, and/or cooperatively implemented by a mobile application implemented on the user/patient's mobile device using the mobile device's native GPS, cellular and/or Wi-Fi triangulation or like self-localization technology, can yield enhanced medical screening and tracking capabilities. For example, not only can a given patient gain access to cumulative knowledge and updates for the purposes of self-screening against dynamically optimizing medical baseline profiles, population mobility, interactions and symptom tracking and monitoring can be implemented in real-time or near-real-time with high physiological data and medical screening accuracy, and high geographical localization and mobility accuracy. As detailed further below, such self-localization capabilities may thus provide for greater oversight on geographical infection rate or symptomatic distributions, migrations, evolutions or mapping, while also geographically mapping treatment effectiveness, recovery or the like.

Such integrated medical and geographic information, optionally combined with demographic or similar patient-related data, may provide particularly useful in the context of an outbreak, epidemic or pandemic, or again in monitoring progression of a yearly seasonal flu season, or the evolution and/or mutation and related symptoms or manifested physiological response profiles over time and space of a new virus or illness through geographical mapping, contact tracking, etc.

As detailed further below, within the context of a respiratory illness, such as Covid-19, Severe Acute Respiratory Syndrome (SARS), Middle East Respiratory Syndrome (MERS) or like viruses, which can lead to significant local, regional, national and/or worldwide infection, the acquisition and comparative analysis of data from a large group of individuals in respect of body temperature (fever), blood oxygenation (e.g. blood oxygen saturation, hemoglobin concentration variations, deoxyhemoglobin concentration variations, quantifiable dissolved or stored or metabolized oxygen concentrations or related values, etc.), cardiac activity/health (e.g. heart rate/pulse/pattern) and respiratory activity (respiratory rate/pattern) can result in greater monitoring, but also modeling of the affected group's response to the illness, impact on the group as the illness progresses, improvements or lack thereof in response to different treatments or therapies (pharmaceuticals, herbals, manipulative treatments, posture, life or respiratory support equipment such as ventilators or like oxygen-delivering devices, hyperbaric treatments, intubation, rest, diet, fluid intake, etc.), regional environment (e.g. ambient temperature, relative humidity, etc.), demographics (e.g. age, sex, ethnicity), medical history (e.g. pre-existing conditions, family history, etc.), behavioral characteristics (e.g. smoking or drinking habits, working environment, exposure to adverse substances, exercise habits, lifestyle, etc.) or the like. For instance, whereby a typical medical establishment could produce local results on periodic manual body temperature assessments and recorded blood oxygen saturation results, a more detailed and granular assessment can be provided both in combination from a single user-friendly and cost-effective form factor, but also in isolating, averaging and comparing time-variable profiles for respective (cerebral) arterial and/or venous hemoglobin and/or deoxyhemoglobin concentrations; stored, discharged, dissolved and/or metabolized oxygen content or concentrations derived therefrom; and/or comparative tracking with simultaneously acquired body temperature, heart-related and/or respiration-related parameters. Furthermore, as introduced above and further detailed below, integration with self-localization technologies may provide for automated emergency contact and response capabilities, as well as various epidemiological tracking/mapping capabilities such as infection rate tracking, geographical propagation, evolution and contact tracking before and/or after the onset of defined symptoms and/or designated integrated physiological signal profile. For example, a triggered screening criteria or alert may result from the designated combination of one or more physiological criteria such as, but not limited to, a designated body temperature signature or threshold such as a detectable, mild or severe fever; a recognizable blood oxygen profile (e.g. designated oxidized hemoglobin and/or deoxyhemoglobin concentration variations or profile(s), derived dissolved or metabolized oxygen values or thresholds, blood saturation, etc.); a designated breathing rate variation and/or pattern; a designated heart-related pattern and/or profile; etc. This alert may be triggered locally through self-assessment against preset profiles loaded to the user's mobile device, or again triggered via remote assessment against a global storage or database of such baseline profiles. In either case, the alert may be logged and tracked, along with a geolocation of the user, for geographical mapping and tracking. Reverse and/or forward geographical tracking may also be applied to identifying geo-temporal overlaps with other tracked users so to identify potential infection risks and geographical spread of a particular condition or illness.

In accordance with some of the herein-described embodiments, the epidermal device and related system may be employed to monitor for abnormal blood oxygenation levels, amongst other parameters as noted above, of a user, for example at home and/or exposed to an oxygen-delivery apparatus in a medical or other context, thus in some examples exposing the user to partial oxygen pressures deviating from the normal value of 0.21 atm at Standard Temperature and Pressure (STP) or sea level. Namely, the methods and systems, according to different embodiments, may be used to monitor blood oxygen content (bounded to hemoglobin and/or dissolved in the blood or tissues) and assess accordingly a (escalated) health-related risk of hyperoxia, hypoxia, respiratory illness, contagion, etc. In some of the following examples, according to some embodiments, the user may be subject to utilizing an assistive breathing mask/apparatus, and/or may be located in a sealed and pressurized environment. This may include the user breathing gas that is either a hyperoxic or hypoxic mix (at any pressure) or normal air (e.g. 21% O₂) at a higher or lower pressure than atmospheric pressure.

In some embodiments, the systems and methods described below rely on various oximetry techniques (e.g. pulse or cerebral oximetry, etc.) to identify and quantify the presence of one or more chromophores' molecules in the user's blood. The measured attenuation (or optical density) measured from one or more oximetry probes may be used to derive a corresponding oxygen partial pressure and/or relative oxygen concentration in said blood. This includes, in some embodiments, quantifying the concentration of dissolved O2 (dO2) by monitoring the oxygen input, levels of mixed blood saturation, combined with a physiological model of oxygen transport in the body. Thus, the systems and methods described herein, in accordance with some embodiments, may be used to monitor in real-time a user's health risk of hyperoxia, hypoxia and/or of a respiratory illness, condition or effectiveness.

In some embodiments, the oximetry technique used is based on near-infrared spectroscopy (NIRS). These are based on the fact that distinct biological molecules change their optical properties when binding to oxygen. This phenomenon is caused by the fact that chromophores such as oxygenated hemoglobin (oxyhemoglobin or O2Hb) differs in parts of its absorption pattern from de-oxygenated hemoglobin (deoxyhemoglobin or HHb), and thus in their apparent optical spectrum. These optical differences can be exploited using two or three (or more) distinct wavelengths in combination to measure arterial and/or venous hemoglobin/deoxyhemoglobin concentrations, arterial oxygen saturation, and derived dissolved oxygen values. Visible light penetrates tissue only short distances, since it is markedly attenuated by several tissue components, which absorb or scatter visible light. However, in the near-infrared (NIR) spectrum (ranging from 700 to 1100 nm) photons are capable of deeper penetration of several centimeters or more. Moreover, NIR beams may also penetrate bones, which is prerequisite for trans-cranial cerebral oximetry for example, although generally speaking other probe locations may be used. Aside from the advantage of relatively deep penetration of several centimeters, the NIR spectral region is also characterized by typical differences in the spectrum of oxygenated and deoxygenated hemoglobin, for example. As mentioned above, other chromophores present in the blood that may be monitored using these techniques usually comprise O2Hb and HHb, but other molecules may be monitored as well, for example (and without limitation) cytochrome c oxidase, carbon monoxide (CO), carboxyhemoglobin, methemoglobin, etc.

In some embodiments, it may also be that one or more chromophores being monitored have more complex absorption spectra, such as a broader spectrum and/or comprising of two or more peaks. Generally speaking, the herein described embodiments are not limited to using one to three wavelengths, but may use as many wavelengths as needed to properly characterize the presence of one or more chromophore molecules present in the user's blood. To achieve this, any number of additional wavelengths may be used (e.g. any wavelength ranging from 600 to 1100 nm). Furthermore, measuring such components may result in overlapping spectra features for two or more components. In this case, multivariate statistical analysis methods may be applied to extract a singular signature for each overlapping component. For example, these may include, without limitation: linear (or non-linear) multivariate regression (MVR), principal component analysis (PCA), principal component regression (PCR), discriminant analysis (DA), hierarchical cluster analysis (HCA), soft independent modeling of class analogy (SIMCA), or similar.

Exploiting these natural characteristics for regional oximetry such as cerebral oximetry (or other), a prototypical NIRS probe functions as follows: A light source (e.g., one or more LEDs of different wavelengths) generates NIR light, concerning the spectrum centered around characteristic wavelengths. The emitted beam is directed into the tissue of interest via, in some embodiments, an epidermal probe (patch). The probe is usually attached to the skin above the tissue of interest (e.g. forehead or armpit for optimal concurrent body temperature measurements). Respective stickers of the probes serve to stabilize the probe's position over longer periods, but also restrict entrance of ambient light into the measurement photon pathway. Transcutaneous NIRS is noninvasive and the applied light intensities are not harmful to the tissue, not causing skin burns even if applied for a longer period.

Generally, the change in molar concentration of the monitored chromophore (for example O2Hb or HHb) may be calculated from the measured change absorbance/attenuation of the NIRS signal by using a physical model of light diffusion and attenuation in organic tissues derived from radiative transfer theory (e.g. using a modified Beers-Lambert law or similar; for example see: Susumu Suzuki, Sumio Takasaki, Takeo Ozaki, and Yukio Kobayashi “Tissue oxygenation monitor using NIR spatially resolved spectroscopy”, Proc. SPIE 3597, Optical Tomography and Spectroscopy of Tissue III, (15 Jul. 1999); doi: 10.1117/12.356862 and Michael S. Patterson, B. Chance, and B. C. Wilson, “Time resolved reflectance and transmittance for the non-invasive measurement of tissue optical properties,” Appl. Opt. 28, 2331-2336 (1989), the entire contents of each of which are hereby incorporated herein by reference).

Furthermore, in some embodiments, the molar concentration may then be used to assess, for example, the dissolved oxygen content in the user's blood (dO2). Indeed, oxygen is found in two forms in the blood: in solution (or dissolved) and bound to hemoglobin. Since dissolved oxygen may accumulate in the blood and be discharged at a later time when partial oxygen pressures are lowered, such an assessment may be important for assessing a user's risk level. In some embodiments, a physiological model may be used to calculate the concentration of dO2 (or change thereof). For example, such a model may determine, in some embodiments, the component or fraction of the inhaled oxygen (e.g. as breathed in directly or via an oxygen delivery apparatus) that is absorbed into the blood stream from measurement of the partial pressure of the inhaled gas mix since the inhaled and absorbed gases will reach equilibrium across the alveolar-blood interface. Current knowledge of physiological processes (such as Fick's diffusion law, O2 solubility, etc.) allows for this evaluation. For example, assuming that the oxygen that enters the blood stream is either bounded to hemoglobin or remains in a dissolved state, changes in the oxy and deoxyhemoglobin concentrations in the target mixed blood volume can be evaluated. This in turn allows for a determination of the component that remains in the dissolved state under the assumption that other physiological parameters such as total hemoglobin number, blood volume, etc., remains nominal and constant. In some embodiments, such a model may use additional parameters such as the oxygen intake (e.g. quantity of oxygen inhaled) for example derived from a measurement of the flow rate of the inhaled gas mix, ambient pressure, an estimation or measurement of the user's blood volume, etc. In some embodiments, an index of user dO2 levels may be constructed for reference.

Other user body parameters may be used, for example and without limitation, parameters related to the user's weight/height, age and/or physical fitness.

In some embodiments, the monitoring systems and methods described herein may further be used to derive a cognition level or index of the user at different levels of blood oxygenation (bounded to hemoglobin and/or dissolved in blood or tissues). The cognition level may include characterizations of user fatigue, stress, confusion, engagement, workload and may be used to assess the ability of the user to concentrate and/or accomplish different tasks (e.g. efficiency and precision), such as diving, piloting an aircraft or spacecraft, etc. The systems and methods described herein, in some embodiments, may further display the user's cognition level in addition to health-related (escalated) risks of hyperoxia, hypoxia, respiratory illness and/or contagion. The cognition level may be derived, for example, by initially assessing the user's ability to execute specific tasks (i.e. speed of execution, number of errors, etc.) while monitoring changes in blood oxygen levels and deriving correlations from those measurements. In the case where the user's cognitive level is determined to be below a certain safety threshold for performing a specific task (i.e. flying an aircraft, etc.), the user may decide or be forced to stop and/or take a break.

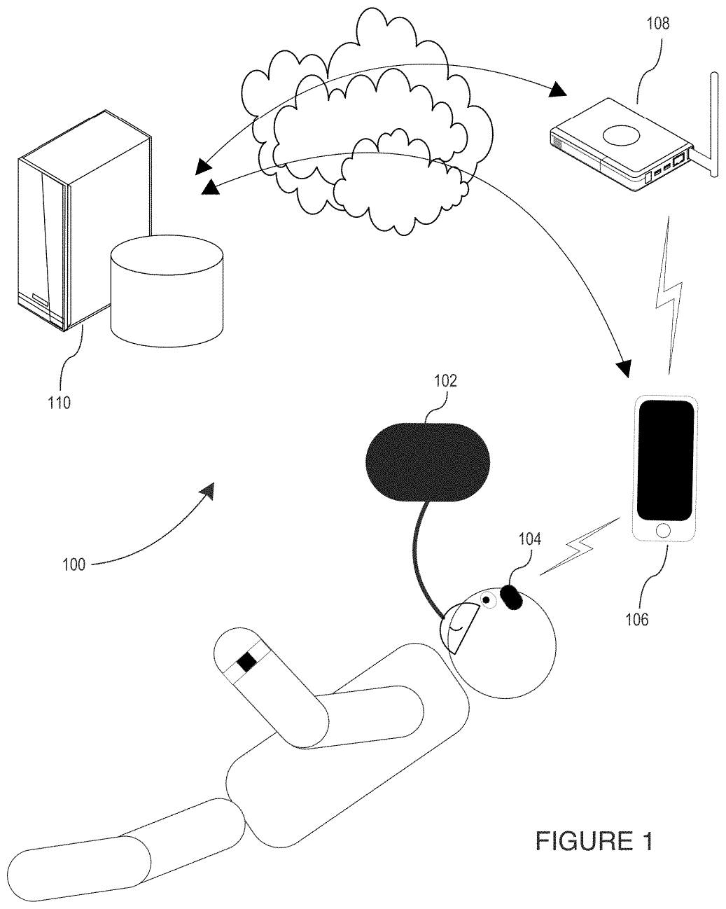

With reference to FIG. 1, and in accordance with one exemplary embodiment, an epidermal health-related sensor and monitoring system, generally referred to using the numeral 100, is shown. In the illustrated embodiment, the system 100 is configured to monitor for abnormal cerebral blood oxygenation levels, an increase or variation in body temperature (e.g. fever) and other health-related parameters illustratively accessible via epidermal cranial spectroscopy such as heart rate, pulse, blood pressure and/or respiration rate. In this exemplary embodiment, the patient is operating an oxygen delivery or breathing-assistance apparatus 102, such as a ventilator, etc., though other oxygen-providing devices or means may be considered, for example.

In this embodiment, the system 100 generally comprises at least one integrated epidermal patch 104, itself comprising a near-infrared spectroscopy (NIRS) probe and electric body temperature probe (neither explicitly shown), fixable through epidermal adhesion in this example to the user's forehead for acquiring cerebral blood oxygenation data, body temperature data, and related data for downstream processing. As mentioned above, other embodiments may be configured to monitor different chromophore molecules present in the blood, such as deoxyhemoglobin and/or oxygenated haemoglobin levels/concentrations, without limitation.

In some embodiments, the integrated patch 104 may be integrated inside a type of headwear, such as a headband or cap. In this case, the headwear should be solidly affixed on the head of the user to avoid suboptimal measurements due to a suboptimal contact between the various probes and the user's skin. As will be discussed below, other embodiments may use different skin contact locations, for example and without limitation, the neck region (e.g. through adhesion and/or a collar). In some embodiments, the patch 104 will include a single-use or rechargeable battery or like power source so to power operation of the integrated sensors and communication means. In one such embodiment, the patch's power source may be rechargeable via a wired link, or again wirelessly charged by induction. In another embodiment, the patch may include a wired power port continuously or periodically powered or recharged from the user's mobile device. In yet another embodiment, single use patches may be operated for a defined lifetime, and replaced thereafter with another patch.

From the absorption spectra measured from this patch 104, a relative cerebral (or regional) blood levels of these proteins may be calculated. To do so, the at least one patch is operatively connected to a digital data processor 106 programmed to compute the relative concentrations of both O2Hb and HHb, and/or a change in molar concentration of HHb or similar. In this embodiment, data is transferred through a wireless connection, but other embodiments, such as wired connections, may also be employed. As will be explained in more detail below, the digital data processor 106 is further programmed to use these relative concentration measurements to derive or define, either locally or through communicative access to a remote monitoring/processing system, at least a lower or higher health risk rating.

It will be appreciated that the processor 106 may take various forms, which may include, but is not limited to, a dedicated computing or digital processing device, microprocessor, a general computing device, tablet and/or smartphone interface/application, and/or other computing device as may be readily appreciated by the skilled artisan, that includes a digital interface to the patch 104 output so to acquire and ultimately process readings/spectra captured thereby. In the illustrated example, the processor 106 is provided by a user's portable device such as a smartphone communicatively linked to the patch via a wireless link, such as via Bluetooth™ pairing, Wi-Fi, NFC or other local short or medium-range wireless communication protocols. While local processing may be implemented on the user's portable device 106, directly, in the illustrated embodiment, a further communicative link is made either directly (e.g. via cell data link) or indirectly via a local router 108 (e.g. local Wi-Fi or LAN), to a remote server and database 110, wherein acquired data may be relayed thereto for processing, and/or to fuel global data processing efforts to track, update or optimize global treatment or illness data and provide improved assessment benchmark profiles (e.g. global averages, trends, recovery patterns, illness escalation patterns, etc.) so to concurrently improve global reporting but also improve treatment protocols and/or profiles, diagnostics, testing, screening or the like.

Furthermore, the embodiment of FIG. 1 further comprises a digital user interface, in this case provided via mobile device 106, capable of displaying a health risk indicator to the user in response to such data processing against benchmark profiles. In other embodiments, the digital user interface and digital data processor 106 may be provided by distinct devices, for example, as will be readily appreciated by the skilled artisan, as can various health-risk-escalating alerting or reporting systems be operatively linked thereto for use by family members, caretakers, or the like. In some embodiments, such digital user interfaces may be comprised of a computer with a digital display screen, tablet, smartphone application or like general computing device, or again a dedicated device having a graphical or like general computing device.

In some embodiments, additional sensors may also be used in parallel or integrated with the epidermal patch 104. For example, pressure, temperature sensors (and/or one or more same and/or distinct physiological sensors or like components operable to interface with the user (e.g. via a direct or indirect user contact, such as a skin contact or like interface operable in contact with or in close proximity to the user's skin or body) may also be used to acquire environmental and/or physiological signals and operatively connected to the digital data processor 106, either for direct transmission consumption or to be used as additional input in the determination of the user's higher or lower health risk rating. Examples of physiological signals that may be monitored via one or more physiological sensors include, without limitation, electrocardiograms (ECG), electroencephalograms (EEG), breathing rate, VO2, blood pressure, body temperature, etc. As will be discussed below, in some embodiments, one or more physiological signal may be correlated with the NIRS probe signal to provide a more precise quantification of blood oxygen levels, amongst other assessments.

In some embodiments, user body position may also be monitored with one or more accelerometers (not shown), as the user body position may affect the flow of blood to the head region (as will be explained below) and thus the spectral response, and/or as such position may impact escalation, assessment and/or treatment for a particular respiratory illness or condition. Therefore, in some embodiments, system 100 may further comprise one or more accelerometers communicatively linked to digital data processor 106 to detect changes in user body position or orientation (e.g. sitting vs. supine, moving vs. sedentary, etc.).

In some embodiments, system 100 may further comprise an internal memory or data storage module (not shown) communicatively linked to digital data processor 106 to store additional data which may be used to improve the monitoring capabilities of system 100. For example, and without limitation, a local or remote (110) spectral database comprising information about the spectral signature of one or more known chromophores may be stored therein.

In some embodiments, digital data processor 106 and/or server-based processing 110 may further be configured to provide additional features, such as an artificial-intelligence-based monitoring system. In some embodiments, the system may be configured to run an artificial intelligence (AI) program to provide user-specific automated or semi-automated oxygen, temperature, respiratory, cardiac and/or related symptom monitoring, as will be explained below. For instance, AI can be integrated so to dynamically learn specifics for each given user and allow for downstream personalization of monitoring and alarm thresholds, for example.

Furthermore, digital data processor 106 may also, in some embodiments, be communicatively linked to the oxygen providing apparatus/device 102 so as to regulate the flow of gas to the user, depending on the user's blood oxygen levels being monitored, overall health condition, status of respiratory illness, perceived effectiveness of current oxygen delivery and/or treatment protocol, etc.

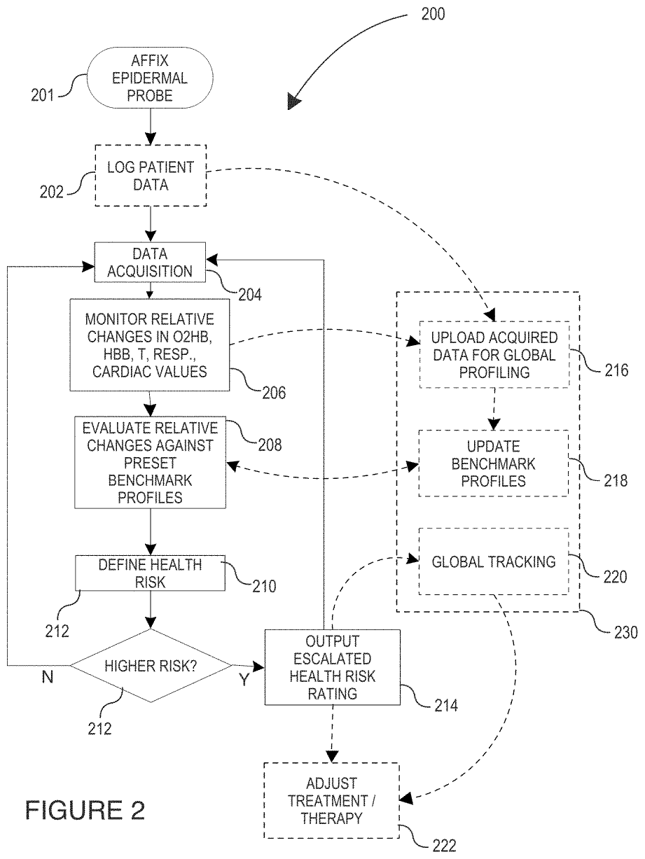

With reference to FIG. 2 and in accordance with one exemplary embodiment, a method 200 for operating the system 100 of FIG. 1 will now be described. The user first affixes (step 201) the integrated (cerebral NIRS/body temperature) epidermal probe to, for example, his/her head. The user/patient then optionally logs user data (step 202) for local or remote tracking purposes (name, demographic, medical history, medical condition, etc.).

The patch acquires data at step 204 to monitor for relative variations, for example, in oxyhemoglobin (O2Hb) and/or deoxyhemoglobin (HHb) levels or concentrations, body temperature, heart rate, respiratory rate, blood pressure, etc. This data acquisition is done continuously, in real-time or at short intervals. In the presently discussed embodiment, the acquired data is analyzed locally by monitoring for relative changes in O2Hb and HHb levels (step 206), amongst other data variations as noted above. These relative changes are automatically evaluated against present variations corresponding to a plurality of benchmark health related profiles, such as cerebral blood oxygenation profiles, temperature thresholds or benchmarks, etc. (step 208). These profiles are preset and/or updated through a remote central server interface, as described above. As mentioned above, the profile may further comprise, in some embodiments, data related to one or more physiological signals, which would be acquired concurrently using one or more physiological sensors. Furthermore, as discussed before, the profile themselves are associated with a preset health-related index that defines at least a lower health risk rating and a higher health risk rating (step 210).

As mentioned above, method 200 may also use at step 210 an artificial-intelligence-based system to provide an improved monitoring capabilities of the user's oxygen levels and related risks. Such a system may receive and analyze in real-time any data being acquired via the patch, one or more physiological sensors, user-body parameters, total oxygen intake, manual changes in the oxygen content flow rates, etc. Different AI, machine learning and/or system automation techniques may be considered to implement such a program. For example, these may include, without limitation, supervised and/or unsupervised machine learning techniques, linear and/or non-linear regression, decision trees, etc. Deep learning algorithms may also be used, including but not limited to, neural networks such as recurrent neural networks, recursive neural networks, feed-forward neural networks, convolutional neural networks, deep-belief networks, multi-layer perceptrons, self-organizing maps, deep Boltzmann machines, and stacked de-noising auto-encoders or similar. As such, the intelligent monitoring features may operate autonomously or semi-autonomously, with limited or without explicit user intervention.

Using hyperoxia as an example, if the method determines at step 212 that the user is experiencing a lower risk of hyperoxia, nothing is done and the method continues the process of acquiring data of step 204. In contrast, if the method determines that the user is currently experiencing a higher health risk of hyperoxia, the method then outputs the higher health risk rating to the user (step 214) to inform him/her of the higher risk so that he/she may take action to reduce it. Risk or condition escalation may also be used to locally induce or recommend adjustment of a treatment or therapeutic protocol (222), as the case may be, so to dynamically monitor an impact thereof on the user's condition/wellbeing. Different examples of indicators may include, but are not limited to, visible indicators such as flashing and/or coloured lights, audible alerts (e.g. relayed through a communicatively-linked earpiece), vibratory device, or the like, which may take the form of continuous, blinking, pulsing, rhythmic, periodic and/or escalating alerts indicators. In some embodiments, visual indicators may be shown on a digital display or like device.

Then, as in the previous case, the method continues the process of acquiring data (step 204).

In this embodiment, optional tracking and feedback may be provided by a remote server system 230 operable to receive logged patient data 202, upload acquired user (raw, preprocessed or fully processed) health-related data or relative changes therein for global profiling and/or tracking (216), optionally use this data to update, refine or optimize benchmark profiles (218) can be relayed to/exchanged with the user's local device/system, and also serve to globally track health risks, conditions, improvements, etc. (220) and ultimately serve to update treatment/therapeutic protocols (222).

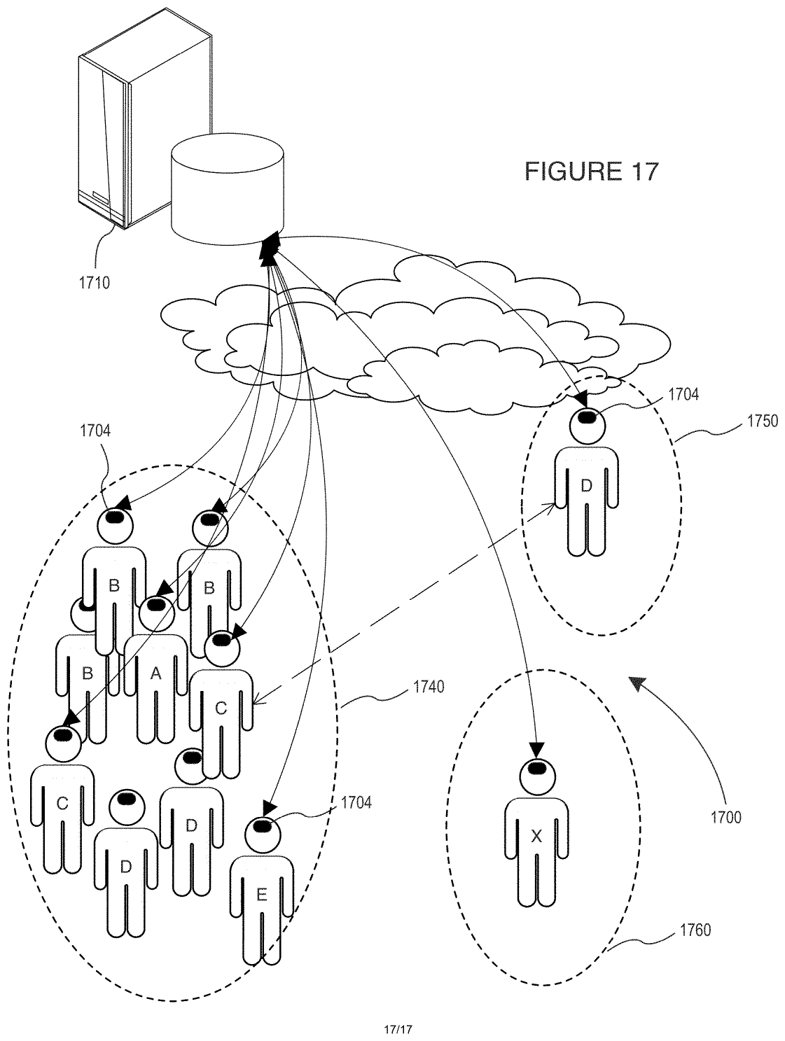

With reference now to FIG. 17, and in accordance with another embodiment, a geographical or epidemiological health monitoring and tracking system, generally referred to using the numeral 1700, will now be described. As will be appreciated by the skilled artisan, a geographical system should be understood to be deployable across a variety of geographical areas, such as, but not limited to, a municipal, regional, county-wide, provincial, state-wide, national or international area, or even globally, with option to subdivide tracking, monitoring and reporting capabilities according to different underlying areas ranging from neighborhoods and communities, to nations and beyond.

In the illustrated embodiment of FIG. 17, the system 1700 relies, at least in part, on vital or symptomatic data acquired or otherwise captured by one or more health related sensors disposed so to actively track a health-related condition of its various participants, wherein such participants may include, but are not limited to, volunteers, patients or participants in one or more schools, workplaces, hospitals, health care of institutional settings, or again volunteers or mandated individuals in one or more prescribed communities, areas, etc. For descriptive purposes, the system 1700 in FIG. 17 is illustrated to interface with epidermal sensors 1704 much as those (104) described above within the context of FIG. 1, for users with or without support from an oxygen-delivering apparatus. In some embodiments, however, the system 1700 may be further or alternatively configured to operate with other dedicated and/or common network-enabled health-related sensors, such as, but not limited to, medical grade or institutional oximeters, cardiac monitors, thermometers or the like, or again, common or dedicated wearable sensors such as those previously operated within the context of a smartwatch, fitness tracker (e.g. integrating various health-related monitors in concert with integrated GPS and/or Bluetooth™ pairing capabilities). In some embodiments, the system may interface with different types of sensors, or require deployment of dedicated sensors for the purpose of data integrity, reliability, and/or user privacy considerations.

Within the context of FIG. 17, a sensor 1704 worn or otherwise linked to a first infected user “A” can report, or result in the remote identification of a set of designated health-related parameters corresponding with a designated condition or illness, which result is monitored and tracked by global server system 1710. Given self or remote-localization capabilities, the first infected user “A” can be geographically identified in area 1740. Over time, other infected users “B” may be identified in geographic area 1740 suggesting a local spread of the condition or illness in that area, and encouraging further regional measures to contain this spread as it continues to spread to users “C”, “D” and “E” over time in this region. Using this system and various medical, geographical and/or mobility modeling techniques, this infection spread can be linked and traced back to user “A” with reasonable accuracy, particularly where similar symptoms and/or medical health-related sensor data patterns or profiles match to a significant degree. Other parameters such as demographics, medical history, etc. may also be considered. Furthermore, by relying on comprehensive medical data analysis, one may observe an invention spread geographically despite some intervening users not exhibiting otherwise readily perceptible symptoms (e.g. an asymptomatic user may still carry the illness and manifest certain health-related signatures observable by the system).

In the illustrated embodiment, the spread of the infection to other areas, such as region 1750, can also ultimately be observed and traced back to a user in region 1740 upon recognizing that an isolated infection in region 1750 likely resulted in that user having recently interfaced with a user “C” in region 1740 (e.g. should a location of a user from region 1740 have previously overlapped within a designated timeframe (e.g. carrier or incubation window) with a similar location from the infected user of region 1750. This can thus not only identify infection spread, but also potentially identify infection routes and/or causes, which can be used to revise or improve infection containment measures. As noted above, variations and/or evolutions of reported or measured symptoms or vital signs may also educate the system on an evolution or mutation of the illness, and approaches to treating it (e.g. observing greater treatment effectiveness or rehabilitation in some areas over others).

Yet in the same example, another isolated event “X” in region 1760 can be further investigated should there be no geographical overlap with other current or previously infected users, suggesting other means of transmission and reason for further external investigation.

Accordingly, using reliable health-monitoring sensors and designated multivariate health risk profiles, in combination with geolocational tracking and reporting capabilities, greater epidemiological data can be captured, shared, and acted upon to reduce the spread of an infection, expedite treatment and/or isolation of infected parties, and/or evaluate containment or treatment strategies.

The person of ordinary skill in the art that the above examples may be implemented using different techniques, equipment, and protocols, and that all such variants should be considered to fall within the general scope and nature of the present disclosure.

Likewise, the above-described embodiments may be applied to different contexts or applications, such that, for example, user health-related tracking and monitoring (e.g. monitoring for illness, heat stroke, dehydration, fatigue, etc.) may be applied in an open or enclosed space, for example, in or across multiple (large scale) industrial or manufacturing settings, office towers, hospitals, retirement residences, long haul cruise or cargo ships, mining centers, amusement parks, shopping malls, farms or like institutional, recreational and/or workplace environments. These and other such examples should be considered to fill within the general context of the present disclosure.

The following provides various other details/examples applicable within these contexts.

As noted above, some of the methods and systems described herein make use of vital sign monitoring devices, optionally using non-contact (at-a-distance) and/or contact (wearable) platforms, for users optionally using or subject to using self-contained oxygen delivery systems or oxygen supply systems used in treatments such as in hyperbaric therapy and supplemental oxygen therapy. Enhanced blood oxygenation monitoring capabilities for tracking risks of hypoxia and/or hyperoxia are also considered, in some embodiments, with further tracking capabilities to identify both abnormally high and low levels of oxygen and detect when a user has reached hazardous levels. In one embodiment, spatially-resolved spectrometry can be used to derive absolute tissue saturation in addition to pulse and respiration rates. Inertial Measurement Units (IMU) can also be used to account for motion of the subject being monitored.

To leverage the benefit of access to large amounts of continuous data, in some embodiments, the device and system is also enabled with an Artificial Intelligence (AI) capability which enhances its performance at identifying specific conditions of concern, and allowing it to be personalized for each user's physiology.

While specialized use in considered in some embodiments, other embodiments may be adapted to vital sign monitoring for a wider general population. For example, as detailed above, single detector oximetry (as opposed to spatially-resolved spectrometry) can likewise integrate complementary vital sign sensors and at the same benefit from access to Big Data.

One exemplary sensor design comprises several subsystems, each handling a core function including data acquisition, data storage and processing, power supply, communication, inertial measurement (IMU), and positioning.

In this example, the data acquisition subsystem is in contact with the skin. It carries the LEDs and detectors. The design uses a medical-grade adhesive foam to keep the device attached to the skin while blocking out ambient light. This adhesive layer is disposable. The rest of the module is sealed and can be disinfected.

The data processing subsystem currently works in two modes. The offline mode is used when measurements need to be stored for post-processing because no stable real-time communication is possible, such as when the subject is underwater or out-of-range to a receiving station. The offline mode can also be used in medical situations where the patients is mobile and requires monitoring without continuous access to the data acquired by the device. When communication is possible, the module can be run in an online mode with live data transmission to a base station (e.g. mobile device, router, watch, or the like) for real-time processing and monitoring. In either mode, the module is programmed to trigger an alarm when conditions pre-defined as hazardous are detected.

The power subsystem carries the rechargeable battery and is integrated with the rest of the device to allow for a one-piece compact device. In the current prototype, the option exists to have power relayed to the sensors through a wired connection to allow for prolonged runtime with larger batteries packs.

The communication subsystem is currently designed with Bluetooth BLE connectivity. From our testing to date, we have shown that the BLE protocol is sufficient for the required data transfer and allows the maximization of power efficiency. Bluetooth allows for a broad range of compatibility with COTS tablet interfaces and network computers.

The IMU subsystem can be used to track motion of the subject. The data can be used to compensate for motion as well as to detect the patient's orientation that can be a factor when calculating blood flow and volume, for example.

The positioning subsystem uses high-accuracy ultra-wideband (UWB) positioning technology. The positioning using this approach uses a dedicated module that communicates with base stations. Positioning through GPS, either via an integrated GPS receiver or via an integrated GPS of a paired mobile device, can otherwise be used, as noted above. UWB nonetheless provides some advantages over GPS in some environments, such as indoors, where a very significant portion of medical applications are expected to be. Yet, depending on the granularity of the localization data required, different levels of self-localization technology may be considered.

In one embodiment, the device is capable of measuring oxygen saturation levels and pulse rates and respiration rates; it comprises integrated, or interfaces with complementary accelerometers to track and report motion; and it is compatible with positioning systems. In one such embodiment, the device is Bluetooth-enabled and designed to communicate directly with either a COTS tablet or PC.

As noted above, the device can be designed with an integrated BLE chip to connect to mobile devices and PCs that will act as base stations for command/control and data exchange.

In some embodiments, the device is designed to be waterproof (to technical diving depths) and can withstand extreme environments with high and low atmospheric pressures, should it be necessary.

It is also designed with a built-in clock for timestamping data and synchronisation, and current power specifications are set at roughly 48 hours.

In some embodiments, as noted above, different powering techniques may be considered. In one example, an integrated battery is provided in the main housing of the device to reduce size and increase wearability and versatility. In another example, an independent battery housing can be connected by wire to the main module for increased autonomous operational time. Power consumption will depend greatly on the frequency of data acquisition, transmission, processing, etc. The current design calls for autonomous operations for at least 48 hours in nominal mode. This can be adjusted to meet the needs of a particular application.

In some embodiments, the device is small and meant to be worn while conducting arduous tasks (underwater, military piloting, firefighting, etc.), and is therefore suited for wearing when exercising and sleeping.

Other aspects of different embodiments include different placement options, such as the forehead in one example to target oxygen levels in the cerebral region, or on the neck to target measurements in the carotid artery. Other body locations can also be considered.

As noted above, either of spatially-resolved spectrometry or single-detector oximetry can be considered, as can other options. Spatially-resolved spectrometry generally involves multiple detectors at defined distances from a light source. The increased number and type of sensors, increased processing and data requirements, as well as power requirements represent an increased cost. However, this in turn offers unique functionality that can make the device optimal for versatility in operation and richness of the data acquired. A variant with one light source and one detector can otherwise be used, for example, for transmission or reflectance measurements, the latter significantly improving the flexibility of positioning of the device as it could be anywhere on the body whereas transmission-type sensors are typically limited to the fingers, toes, or ears (with drawbacks as a noted above).

As described above, a miniaturized temperature sensor specifically designed for body temperature measurements can be used in concert with oximetry and/or other sensors.

Blood pressure can also be measured through photoplethysmography or other sensor techniques. The noted technique generally relies on recognizing similarities between arterial blood pressure (ABP) and photoplethysmography (PPG) signals, noting that PPG signals contain much of the information that is used to derive blood pressure from conventional ABP signals.

As further noted above, some embodiments may be configured to implement an AI-enabled predictive algorithm and a geospatial-vital sign trending feature. Both features are possible given the unique capability to acquire Big Data with this device. For example, large-scale vital sign monitoring data can be acquired with continued use of the device. This data can be used to assess trends in data variability and train each device with a predictive ability. This feature will also allow a comparative analysis of the trend observed for one individual in relation to the population-based datasets.

The opportunity to have a widely distributed vital sign monitor that can be positioned offers the unique opportunity to capture epidemiological data on a real-time scale that has not been previously possible and can be especially useful in pandemic scenarios to study disease spread. Geospatial trends can be analyzed without the need to mobilize large populations to enter data, without risking false or erroneous data, while ensuring data anonymity.

The following explains, in part, how cerebral blood oxygenation profiles may be determined, in accordance with one embodiment, to go beyond standard blood oxygen saturation results and thus, provide for greater granularity and flexibility in evaluating a patient's oxygen intake, metabolism, overall respiratory and/or circulatory health, and/or related conditions, health factors or illnesses. The person of ordinary skill in the art will readily appreciate that while such examples are provided, different embodiments of the herein-described health-related sensors, monitors, systems and global monitoring, self-learning, and/or tracking systems may also or alternatively rely on other health-related patterns, such as, but not limited to, those presented and described above in various details.

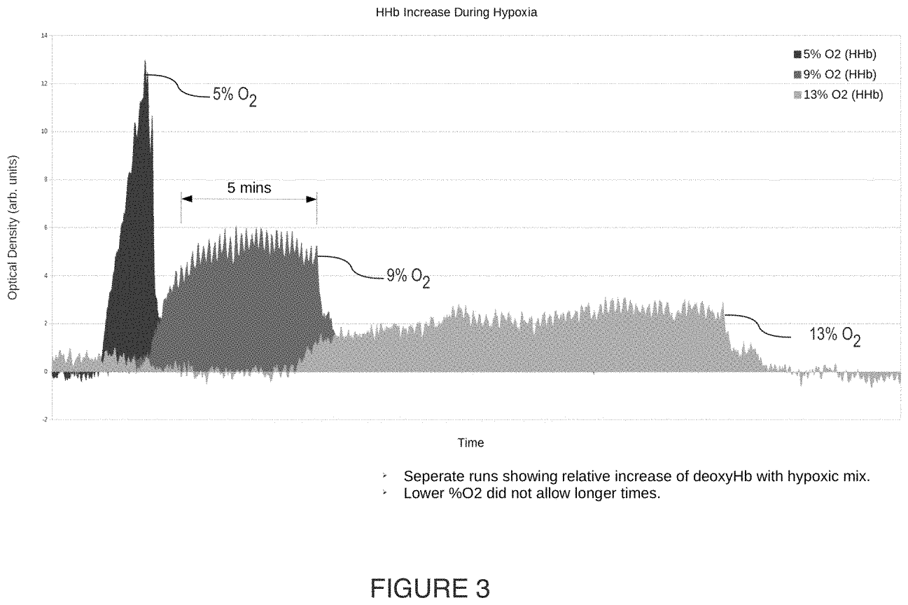

With reference to FIG. 3, and in accordance with one exemplary embodiment, a plot is provided of the relative change in cerebral deoxyhemoglobin (HHb) absorbance (e.g. optical density), as a function of time, of an individual breathing a series of gas mixtures with a reduced oxygen concentration (hypoxic mixes). The measurements were taken using a commercially available NIRS system developed by Artinis Medical Systems B.V. The absorbance values are relative to the baseline values obtained with the same individual breathing normal air (21% O₂) and three runs were measured with hypoxic mixes of 5%, 9% and 13% oxygen respectively. For each data series, the individual sustained breathing the associated mix as long as comfortable, then returned to breathing normal air again. Clearly, breathing lower levels of oxygen, as is well known, leads to a rapid increase in HHb levels. The lower the oxygen level, the faster and higher the rise in measured HHb levels is observed and the shorter the time the individual could sustain respiration.

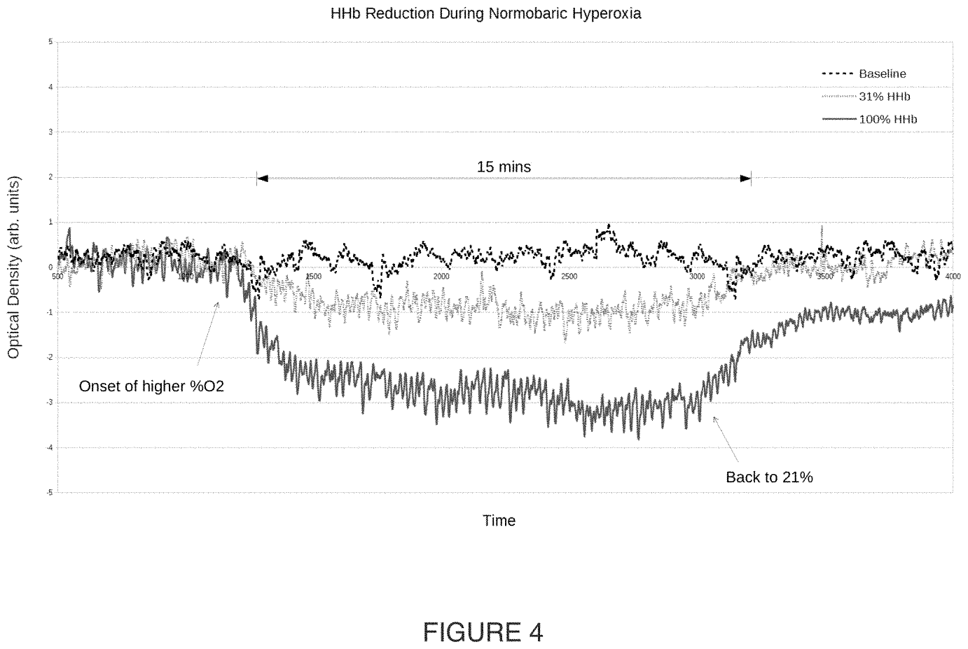

In contrast to FIG. 3, FIG. 4 is a plot, as a function of time, of the change of HHb levels while breathing an increased concentration of O₂ (hyperoxic mix). Three measurements are shown, one baseline measurement at a normal O₂ concentration of 21% (e.g. normal air) (dark gray dotted line), one measurement done with a mix containing 31% O₂ (light gray dotted line) and one measurement with pure O₂ (black line). In the last two measurement series, the individual was breathing normal air in the first and last 5 minutes of the experiment. We clearly see the reduction in HHb concentration measured with the increased intake in O₂. Moreover, while the measurements at 31% O₂ show a quick return to the baseline value after the individual stopped breathing the gas mixture, in the second case, while the HHb concentration increases and stabilizes after the pure O₂ is removed, it never quite returns to baseline during the acquisition time, though will clearly eventually return to baseline over time. These measures thus illustrate the tissue's ability to store oxygen, which may become increasingly important for greater oxygen partial pressures. Using methods as described herein, in some embodiments, means may thus be provided to monitor the discharge of oxygen from tissues into the blood.

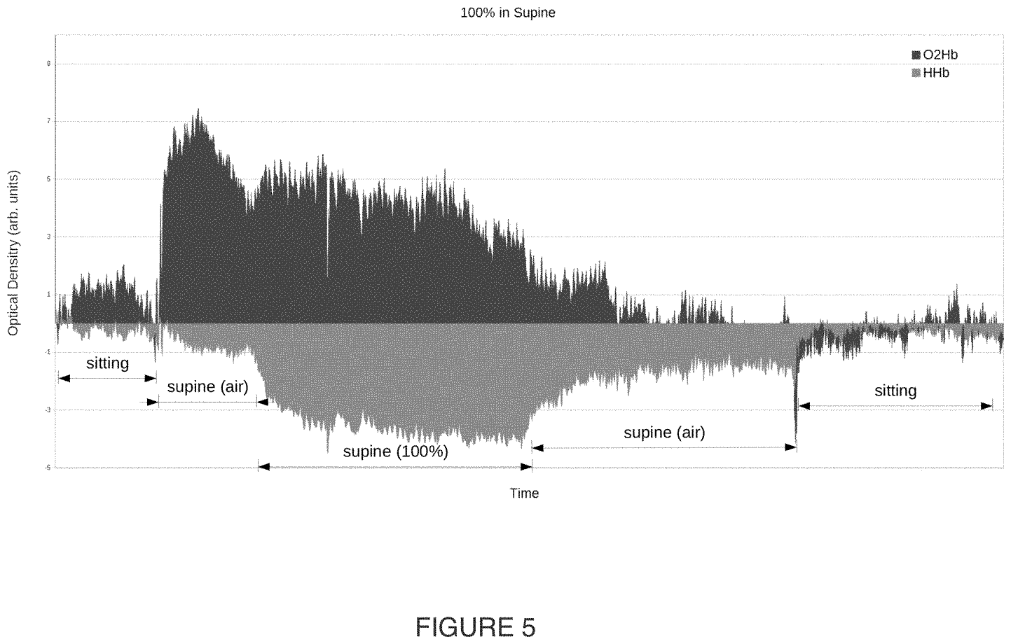

FIG. 5 shows the effect, as a function of time, of both changing an individual's position (sitting or supine) and breathing pure oxygen (100% O₂) vs. normal air (21% O₂). Both the relative absorbance values of the oxyhemoglobin (O2Hb) and HHb are shown. For this experiment, the individual is initially breathing normal air (21% O₂) in a sitting position for 5 minutes, followed by being put in a supine position for another 5 minutes. The individual, still in a supine position, was then exposed to a pure oxygen gas via a face mask for a number of minutes. Without changing the individual's position, the mask was then removed, allowing the individual to breath normal air again. Finally, after waiting a few minutes, the individual was allowed to sit again. We clearly see the effects these changes have on both the O2Hb and HHb measurements. However, we find that the O2Hb and HHb responses are not symmetrical, indicating that measurement only the O2Hb concentration may be unreliable as the only indicator of cerebral blood oxygenation in all contexts. However, a careful measurement of both O2Hb and HHb concentrations using NIRS does lead to the determination of a more precise index.

FIGS. 3 to 5 clearly show characteristic signatures of the changes in O2Hb and HHb levels not only as a function of oxygen content breathed by an individual but also as a function of the individual's relative position. By measuring the changes in O2Hb and HHb levels for a series of different oxygen levels in different individuals, a series of benchmark cerebral blood oxygenation profiles may be recorded. These profiles may then be digitally associated with a preset cerebral blood oxygenation index that may then be used to associate a lower or higher risk rating of hyperoxia in the individual, of example. The benchmark profiles may be expanded to include different partial oxygen pressures by doing measurements inside a hyperbaric or isobaric chamber, for example.

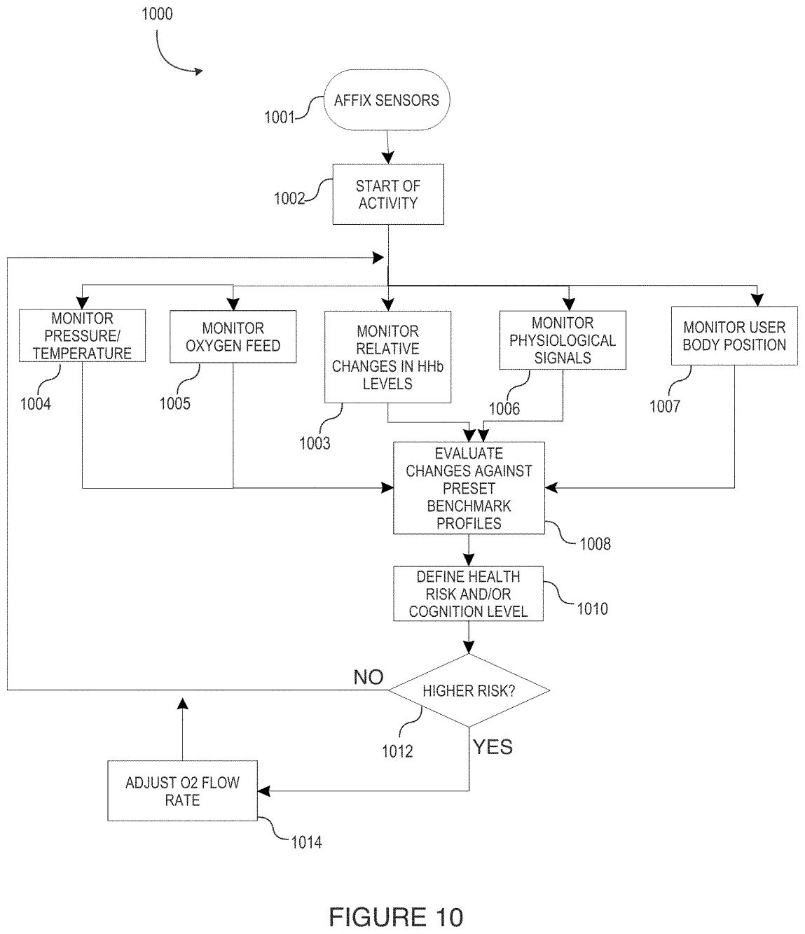

With reference to FIG. 10 and in accordance with one exemplary embodiment, another method for monitoring a user's health risk when partaking in an activity requiring the use of an oxygen providing apparatus and/or inside a sealed pressurized environment, generally referred to using the numeral 1000, will now be described. Similar to the embodiment described in FIG. 2, at step 1001 one or more sensors are affixed or put in contact with the user's skin at one or more locations. These sensors may be integrated into a wearable device as explained above. Once the user begins the activity at step 1002, method 1000 immediately starts monitoring one or more parameters. At step 1003, the method monitors via one or more NIRS probes the molar concentration of HHb in the user's blood (for example in the cerebral region), but may also optionally monitor in parallel other parameters such as ambient pressure and/or temperature (step 1004), oxygen intake (step 1005), one or more physiological signals via one or more physiological sensors (step 1006) and/or user body position via one or more accelerometers (step 1007). Data acquired from steps 1003 to 1007 is sent to a central processing unit (i.e. digital processing unit 106 for example) to be analyzed and compared to preset benchmark profiles at step 1008. As discussed above, in some embodiments, step 1008 may be performed using machine-learning techniques such as deep learning techniques or similar. From this analysis, a health-related risk of hyperoxia/hypoxia and optionally a user cognition level may be defined at step 1010. At step 1012, these risk and/or cognition levels may be compared to previous levels to determine if an increase in risk or a loss of cognition has occurred. In this case, method 1000 may automatically adjust the flow of oxygen delivered to the user to reduce the risk and/or increase the cognition level. Optionally, at step 1014 a warning may also be delivered to the user as explained above. The method then goes back to monitoring different parameters (steps 1003 to step 1007) to assess a new risk and/or cognition level.

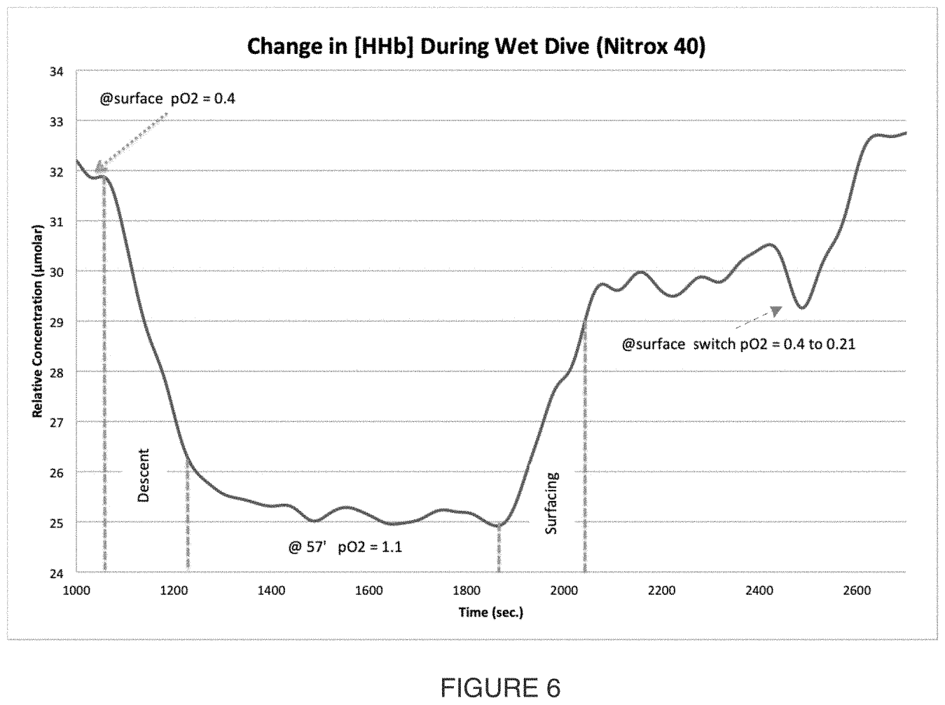

With reference to FIGS. 6 to 9, and in accordance with one exemplary embodiment, different plots are provided of the change in cerebral HHb concentration (in μM or 10⁻⁶ mol/L), as a function of time, of an individual subjected to different oxygen partial pressures. These figures clearly show the different correlations between the measured molar concentration of HHg different parameters, including partial oxygen pressure but also sustained physical activity. The measurements were again taken using a commercially available NIRS system developed by Artinis Medical Systems B. V. Changes in molar cerebral HHb concentrations were calculated from changes the NIRS attenuation signal using the light diffusion model of Suzuki et al. mentioned above. Thus, in FIGS. 6 to 9, only changes in the measured cerebral HHg concentration with respect to the initial value are meaningful and the initial concentration value at the start of each Figure is arbitrary.

For example, FIG. 6 shows a plot of an individual being completely immersed in water at different depths and breathing sequentially from two different gas mixtures (normal air and Nitrox 40 hyperoxic mix). At the start of the plot shown in FIG. 6, the individual is breathing the Nitrox 40 mix while floating at the surface (e.g. p02=0.4). As the depth increases, we clearly see the concentration of cerebral HHb decreasing as well with respect to the initial value (at the surface at t=2000 sec.) by about 7 μM until a depth of 57 feet is reached with a corresponding partial oxygen pressure of 1.1. The diver stayed at that depth for about 10 minutes before resurfacing at around 2400 sec., where we see the concentration of HHb increasing correspondingly by about 2.5 μM, returning it close to its initial value at the start of the experiment. Thus FIG. 6 shows a clear correlation between changes in oxygen partial pressure and corresponding changes in cerebral HHb concentration.

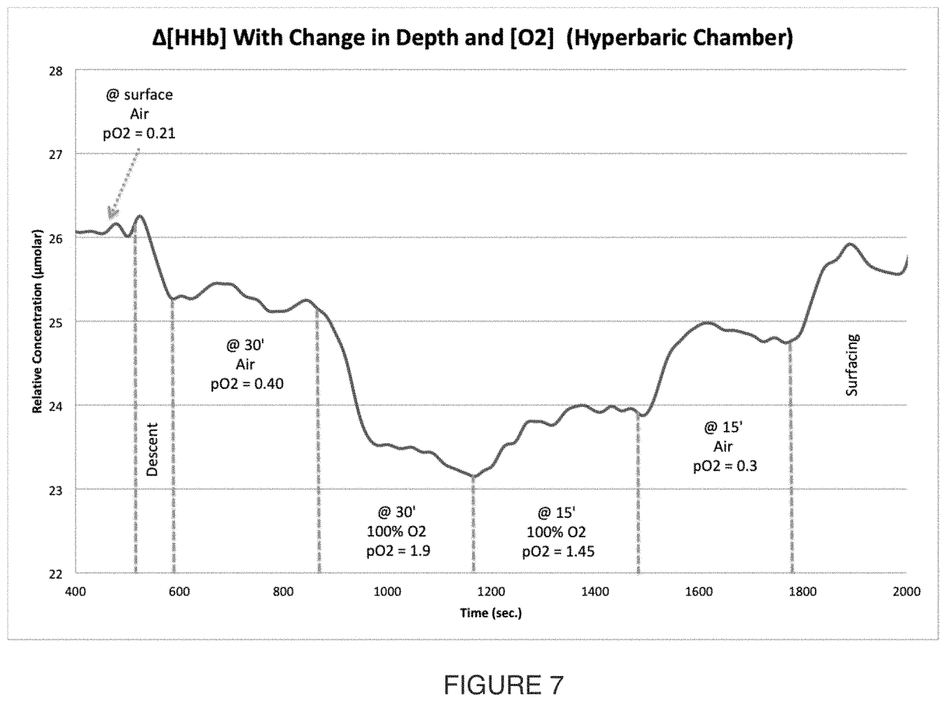

Similarly, FIG. 7 shows a plot of the change in cerebral HHb concentration as a function of time but for an individual inside a sealed hyperbaric chamber where both a change of O2 concentration was administered and a change in depth simulated by varying the pressure. Thus, the partial oxygen pressure inside the user could be changed by either changing the pressure in the chamber or by changing the oxygen concentration the individual was breathing (air or hyperoxic mix). Starting from a normal oxygen partial pressure of 0.21 (e.g. breathing normal air at atmospheric pressure), the pressure inside the chamber was increased to simulate a corresponding depth of 30 feet (pO2=0.40) which led to a small decrease in cerebral HHb concentration (with respect to its initial value att=400 sec.) of about 0.8 μM. Then, at around 900 seconds, the pressure was kept constant but the breathing mix was changed from air to pure oxygen (pO2=1.9). We quickly see the HHb concentration further decreasing by a value of about 2 μM. The next step consisted of letting the individual breathe pure oxygen but to decrease the pressure to simulate a depth of 15 feet (pO2=1.45). We see that this leads to a corresponding increase of the HHb concentration by about 0.8 μM. Then, keeping the pressure constant, the individual was given normal air to breathe (p.02=0.3). We again find a corresponding rapid increase in the concentration of cerebral HHb by a value of 1 μM. Finally, the normal atmospheric pressure was restored which led the concentration to return to its initial value (at t=0). Thus, we clearly see in FIG. 7 the correlation between changes in depth and partial oxygen pressure and the corresponding variations in the cerebral HHb concentrations.

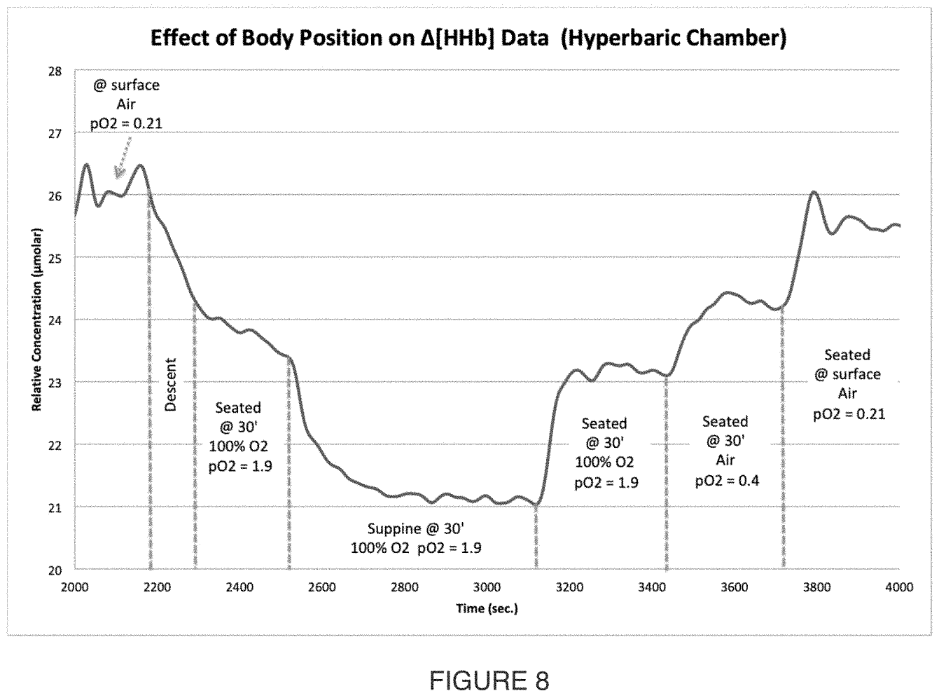

FIG. 8 illustrates, similarly to FIG. 5, the effect, as a function of time, of both changing an individual's position (sitting or supine) and breathing pure oxygen (100% O2) vs. normal air (21% O2), again inside a hyperbaric chamber. In the plot of FIG. 8, the individual is first in a seated position while breathing normal air. The pressure was then increased to a corresponding depth of 30 feet, resulting in a corresponding decrease in the HHg concentration by about 2 μM with respect to its initial value (t=2000 sec.). Then, the individual, still seated, was administered pure oxygen (pO2=1.9) which leads to another decrease of the HHg concentration by about 0.6 μM. Next, keeping the pressure constant (30 feet) and still breathing pure oxygen, the individual was asked to take to a supine position, which causes the measured concentration of cerebral HHb to decrease further by about 2 μM. Going back to a seated position cancelled, as expected, the previous variation by increasing the HHg concentration by 2 μM. Now changing the breathing mix from pure oxygen to normal air (but keeping the pressure constant and a seated position) also returned the cerebral HHg concentration to a value of about 1.9 μM below the initial value. Finally, decreasing the pressure to normal atmospheric pressure returned the measured HHg concentration close to the initial value at the start of the experiment. Thus, we see that the derived molar concentration also correlates well with the user body position.

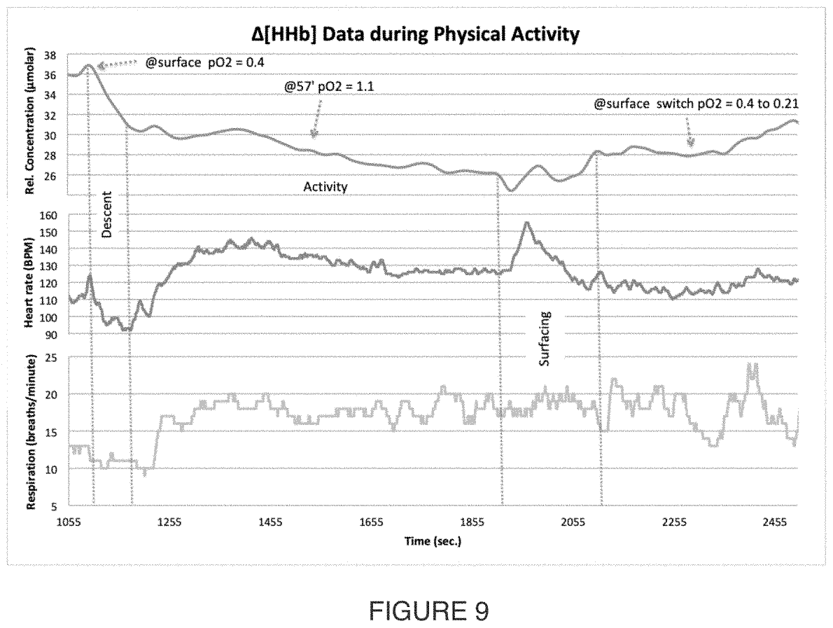

As mentioned above, in some embodiments, one or more physiological signals may be acquired concurrently with the NIRS signal to provide an increased accuracy in the calculated blood oxygen content, for example by using measured correlations between the changes in these one or more physiological signals and the HHb concentration levels (or other chromophores) when the user is engaging in a physical activity. For example, in FIG. 9 we see three plots illustrating the corresponding change over time of the concentration of cerebral HHb, the user's heart rate (in beat per minute or BPM) and the breathing or respiration rate (in breaths per minute), from top to bottom respectively, of a user engaging in an underwater physical activity as a function of time. In FIG. 9, the individual is initially breathing an hyperoxic mix (pO2=0.4) while at rest at the surface and then descends underwater to a depth of 57 feet (pO2=1.1), leading to a corresponding decrease in the measured cerebral HHb concentration of about 5 μM below the initial value (t=1055 sec.). This decrease is also correlated with a small decrease in the heart rate from 120 BPM to about 95 BPM. The individual then started engaging in a physical activity for more than 10 minutes, which immediately results in an increase in the measured heart rate (from 95 BPM to about 128 BPM with peak at 140) and the respiration rate (from about 10 breaths per minutes to around 19-20 breaths per minute), and a corresponding decrease of the HHb concentration by about 4 μM (e.g. 10 μM below the initial value). This decrease in the HHg concentration is directly linked to the physiological processes caused by the physical activity being performed and not linked to the partial oxygen pressure alone, as will be seen below. Following this, the individual returns to the surface while still breathing the hyperoxic mix, which shows as a slight increase in the HHb concentration by a value of about 4 μM. Finally, the diver resumes breathing normal air, which shows up again as an increase in the HHb concentration of about 4 μM. Thus, the measured cerebral HHg concentration at the end is still 4 μM below the initial value at the start of the experiment, which roughly corresponds to the decrease observed when the user was engaged in the physical activity, as expected.

As noted above, conventional oximetry relies on measurement ratios for 2 blood-oxygen-related absorption wavelengths (oxyhemoglobin and deoxyhemoglobin) to produce useable, but limited results. Indeed, absorption ratios lose specificity in observing actual or absolute blood oxygen concentrations and neglect the finer detail otherwise available using techniques as described herein that probe and analyze greater portions of the blood or probe tissue's absorption spectrum. Moreover, since it relies on indices that are derived principally as ratios and are relative to baseline measurements, conventional oximetry also requires calibration (or the use of look-up tables) to associate a measured index with a saturation level, further restricting use and applicability. Furthermore, conventional pulse oximeters often do not provide reliable readings when saturation is low, and require that a pulse or heart beat be continuously and accurately detectable. While spatially-resolved spectrometry can provide further information as it invokes a spatial investigation, it remains constrained to the analysis of relative spatially-resolved concentration ratios, and thus remains unable to extract absolute total concentrations.

In some of the herein-described embodiments, oximetry data can be acquired using a spectroximetry probe, such as that described in greater detail below. For example, the systems and methods described herein provide, in accordance with different embodiments, different examples of a system and method for monitoring or assessing one or more physiological or health-related parameters or condition(s) in a user or patient via full or broad-spectrum oximetry, which is herein interchangeably referred to as spectroximetry or hyperspectral oximetry. Using a spectroximetry probe as described below can, in some embodiments, further enhance implementation of a physiological monitoring system, as described above, though other oximetry probes may also be considered in that context. Meanwhile, a spectroximetry probe and system as described herein may provide other benefits in other contexts, as detailed below, without limitation.

In contrast with standard oximetry, full or broad spectrum oximetry (spectroximetry or hyperspectral oximetry), as provided by the exemplary systems and methods described below, in accordance with different embodiments, has the unique feature of allowing the measurement of the entire absorption spectrum of interest, such as for example between 600 nm and 1000 nm. Namely, a broad range of probing wavelengths can be leveraged, in different embodiments, to extract a coarse or even fine resolution absorption spectrum that carries a greater wealth of information for the purposes of conducing and outputting a more detailed analysis and evaluation of the probed tissue's oxygenation profile, status or condition.

For instance, in some embodiments, spectroximetry has the advantage of measuring absolute energetic transmission over a broad range of wavelength. This absolute measurement can allow for the comparison of absorption profile variations for the same patient at different times, and between patients, for example. Current oximetry methodologies rely on indices that do not lend to such analysis.

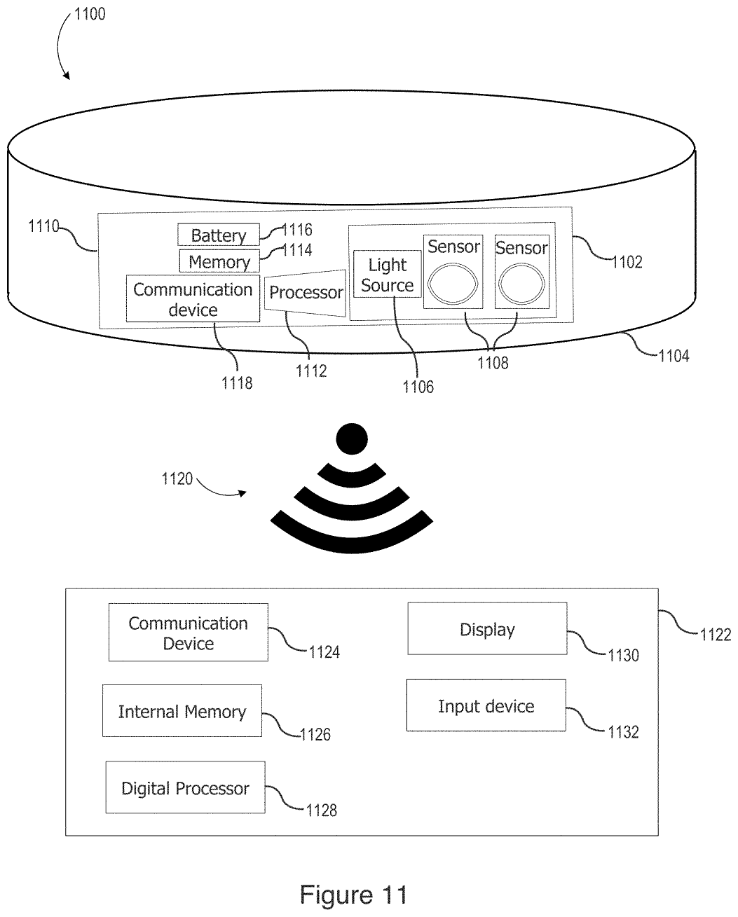

With reference to FIG. 11, and in accordance with one exemplary embodiment, a full spectrum oximetry system, interchangeably referred to as a spectroximetry system, and generally referred to using the numeral 1100, will now be described.

Pulse oximetry is often used in health-related systems to monitor blood oxygenation. This typically works by emitting NIR light into tissues, measuring the corresponding transmitted or reflected light at two distinct wavelengths, and deriving from changes in absorbance a corresponding change in oxyhemoglobin, for instance, in the form of an estimate from relative variations, calibration or via index-tables. In this exemplary embodiment, however, system 1100 comprises a broad-spectrum probe 1102, which may be attached to the skin above the tissue of interest or as illustrated herein integrated into or inside a type of cerebral patch or headwear (here headband 1104). Probe 1102 generally comprises at least one broad-spectrum infrared light source 1106, for example one or more LEDs may be used alone or in combination to generate IR light covering a broad range of the infrared spectrum (e.g. for example wavelengths between 600 nm to 1000 nm). Light source 1106 is generally configured so as to emit light into the tissue of interest, this example the head/brain region. It some embodiments, it may comprise one or more LEDs manufacture into a single device, or in other embodiments multiple LEDs may be used at different physical locations. Naturally, while a broad IR range of 600 nm to 1000 nm is presented here as an example, it will be appreciated upon further reading that different shorter or longer ranges can be considered without departing from the general scope and nature of the present disclosure. It will also be appreciated that different light sources and/or combinations of light sources 1106 may be considered to provide such range, to accommodate different probing spectrum intensity or continuity profiles, or the like, without departing from the general scope and nature of the present disclosure. Furthermore, while focus is made on a broad spectrum IR light source, other complementary spectral regions may also be considered where absorption, transmission and/or reflectance spectra can provide complementary information or characteristics on blood-oxygen or other blood-constituent elements of interest.

In this embodiment, probe 1102 further comprises at least one high-resolution miniature spectrometer or sensor 1108 to record one or more high-resolution absorption or transmission spectra of the transmitted or reflected light from light source 1106. Miniature spectrometer 1108 may take different forms and/or have different specifications. In general, spectrometer 1108 should have a high spectral resolution sufficient to confidently reproduce a representative spectral signature received by probe 1102 over the broadband infrared range of interest. In some embodiments, spectrometer 1108 may be based on a diffraction grating design, a multi-layer filter design, a combination thereof or another design entirely. For example, and without limitation, spectrometer 1108 may be operable to acquire spectral data with a 5 nm resolution over the whole range between 600 nm to 1000 nm (e.g. 10, 40 or even 80 distinct wavelengths/spectral regions). The skilled technician will understand that different numbers of wavelengths with different resolutions may be considered. In general, the acquired spectral data should have a resolution that allows to differentiate between different peaks or dips of interests, with sufficient details so as to allow for comparative analysis of such acquired spectra with designated representative spectra or spectral variations therein, and or with previously or continuously acquired spectra as a user's condition and/or environment changes. Namely, as will be detailed below, acquired spectra may be used for comparative analysis as a single diagnostic or screening tool against preset or designated standard spectra representative of healthy, low risk or high risk conditions, illnesses, and/or environmental scenarios, or again as continuous or regular monitoring means whereby observed spectral profile variations in different spectral regions or combinations of such regions can be quantitatively or qualitative mapped to corresponding conditions or risk factors.

With continued reference to FIG. 11, different configurations of light source 106 and spectrometer 1108 may be considered for probe 1102. In some embodiments, a single light source 1106 and spectrometer 1108 may be used. For example, a single broad IR spectrum LED and a single sensor may be used, with a pre-defined distance therebetween. In some embodiments, sensor 1108 and the single LED of light source 1106 may be placed opposite each other (e.g. with the tissue of interest in-between) so as to measure the transmission (or absorption) spectra. In other embodiments, a linear configuration may be used where the LED of light source 1106 and sensor 1108 are placed next to each other (as shown in FIG. 11), pointing in the same direction, in order to measure the light scattered back from the tissue volume they are placed on.

In yet another embodiment, one sensor/spectrometer 1108 may be placed linearly alongside a light source 1106 comprising multiple LEDs (reflection-type design). In this configuration it may be possible to have different pre-defined distances between each LED and sensor 1108. The difference in distances may thus allow for spatially-resolved data to be acquired.

In yet another embodiment, probe 1102 may consist of a light source 106 comprising a single LED, but with sensor 1108 comprising several individual sensors instead of a single device, e.g. laid out in a linear spatially-resolved reflection-type configuration. This layout thus also allows spatially-resolved spectrometric data with different pre-defined distances between the LED and each sensor.

Going back to FIG. 1, in the illustrated exemplary embodiment, probe 1102 is shown as comprising two detectors 1108 with a single LED infrared light source 1106, placed on or affixed to a mounting platform or casing 1110 that holds them in place on the forehead in proximity to the frontal cortex when the user is wearing headband 1104. While in this exemplary embodiment, a headband is used, the skilled technician will understand that other designs may be used, for example that include smaller patches that can be affixed with medical adhesive or through suction cups. Moreover, other body areas may be targeted with different means of affixing probe 1102 thereto, without limitation.

In addition to probe 1102, platform or casing 1110 of FIG. 1 further contains the electronics and energy source necessary to power and control probe 1102 and communicate to an external computer. For example, this may include a digital processor 1112 communicatively connected to an internal memory 1114, a power source 1116 and a communication device 1118.

Digital processor 1112 may be any type of digital processor known in the art. This may include low-powered microcontrollers, embedded processors or the like. Generally, digital processor 1112 is communicatively linked to probe 1102 so as to at least control its operation and sometimes additionally process, at least in part, the acquired data. Digital processor 1112 is also communicatively linked to internal memory 1114 which may contain for example instructions for use thereby. Internal memory 1114 can be any form of electronic storage known in the art, or a combination thereof, including read-only memory, random-access memory, or flash memory, to name a few examples. Power source 1116 may comprise one or more rechargeable or non-rechargeable batteries. Communication device 1118 may be any device operable to transmit data to another electronic device. This may include a network adapter for transmitting data over a wired (i.e. ethernet) or wireless connection (i.e. Bluetooth or Wi-Fi). It may also include RF emitters/transmitters, for example a wireless UART RF module or similar. In the illustrated embodiment of FIG. 1, communication device 1118 is shown transmitting data via a wireless signal 1120 to a remote processing device 1122. The skilled technician will understand that other electronic components may also be integrated on headband 1104 as required. These may include for example DC/DC converters, or any electronic component required to optimize the functioning of the components already discussed above, without limitation.

In some embodiments, probe 1102 and its associated electronic components may be operable to function in an offline mode in which case the data is stored on board in internal memory 1114 for future download once the wireless link is made available. The device may also function in an online mode when the wireless connection to remote device 1122 is available and can allow real-time download of the data acquired for monitoring and processing purposes.

In some embodiments, remote device 1122 may be any type of a computer with a digital display screen, tablet, smartwatch, smartphone or like general computing device. It generally comprises its own communication device 1124 configured so as to communicate with communication device 1118, an internal memory 1126, a digital processor 1128, some type of display 1130 and one or more input devices 1132 (i.e. keyboard, mouse, touch screen, etc.). In some embodiments, remote device 1122 may be operable to receive spectral data acquired by probe 1102 and to process it.

In some embodiments, as remote device 1122 may not have the constraint otherwise imposed on a wearable probe such as that provided by headband 1104, digital processor 1128 may be more powerful than digital processor 1112 on headband 1104, and may thus be relied on to provide more demanding tasks such as data analysis or the like. In other embodiments, if remote device 1122 is also lightweight (smartwatch, etc.), processing may be offloaded, at least in part, to a remote server or similar (not shown) to which remote device 1122 is or can be remotely connected.

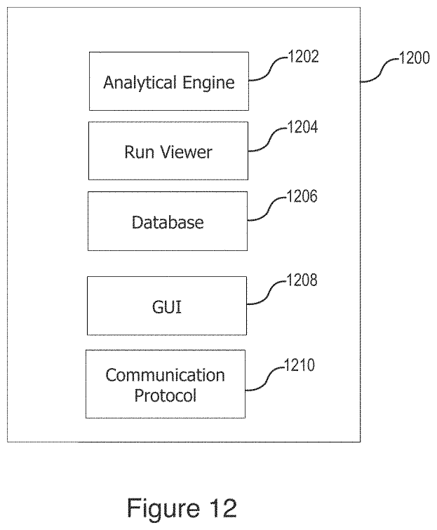

With reference to FIG. 12, and in accordance with different embodiments, a software processing system or engine for processing spectral data, generally referred to using the numeral 1200, is discussed. In this exemplary embodiment, processing system 1200 may be executed on remote device 1122, which is in direct communication with the electronics on headband 1104 as mentioned above. More generally, in some embodiments, processing system 1200 may be in the form of a software interface or application interface running or being executed on a computer with a digital display screen, tablet, smartphone application or like general computing device, or again a dedicated device having a graphical or like general computing device.

In some embodiments, processing system 1200 may comprise one or more software modules or features, including for example an analytical engine 1202, a run viewer 1204, a database 1206, a graphical user interface (GUI) 1208 and/or a headband communication protocol interface 1210.

In some embodiments, analytical engine module 1202 comprises software configured or programmed to process spectral data acquired by probe 1102. This may include fitting the spectral data with one or more spectral functions so as to determine the spectral contributions from one or more chromophores or molecules. It may also include using the identified spectral contributions from each chromophore (and thus a related chromophore concentration) to derive one or more related physiological or health-related parameters. These may include, without limitation, blood volume, blood flow rate, breathing rate, heart rate, and blood pressure and/or any medical condition related to a change thereof. In some cases, this may be done using pre-defined analytical models. In other cases, machine-learning or artificial intelligence (AI) algorithms may be used to derive correlations between these one or more physiological parameters and said spectral contributions. Moreover, by combining an analytical model with the high-resolution spectral data acquired by probe 1102, absolute measurements are possible, in contrast with known methods which rely on relative measurements.

In some embodiments, run viewer module 1204 is a program operable to monitor spectral data acquired via probe 1102, in some cases in real-time. This may include generating plots or graphical representations of said spectral data. In some embodiments, module 1204 may further be used to remotely program or configure probe 1102 or any parameter related to the spectral acquisition process (i.e. acquisition frequency, brightness of light source 1106, etc.).

In some embodiments, database module 1206 may include a database software, or a database-interfacing program operable to interface with a remote server-based database. It may be used to store spectral data acquired via probe 1102 but also any processing done thereto via analytical engine 1202. In some embodiments, previous measurements may be stored in database 1206 so as to construct a baseline for one or more physiological or health-related parameters.

In some embodiments, processing system 1200 may include a headband communication protocol interface module 1210. This may include any software used to configure or control data transmission between probe 1102 and remote device 1122 (or to any other computing device), so for example to configure either one of communication devices 1118 or 1124. In some embodiments, this may also include configuring how other parameters related to the functioning of any components located on headband 1104 may also be transmitted. For example, this may include the remaining charge of power source 1116 or any error messages related to malfunctioning hardware components.

In some embodiments, processing system 1200 may further comprise a GUI 1210, displayed for example via display 1130, and which may be used to interact with any one of modules 1202 to 1208 via a mouse of touchscreen. In some embodiments, multiple modules may be interacted with simultaneously via GUI 1208.

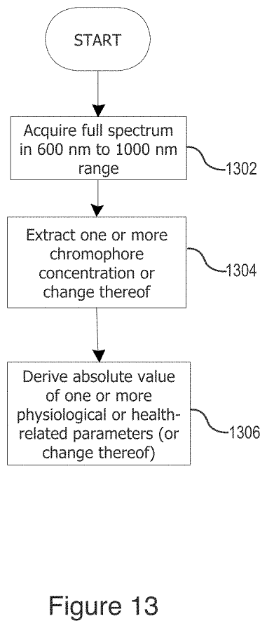

With reference to FIG. 13, and in accordance with one embodiment, a process for monitoring for one or more health-related parameters with system 1100, generally referred to using the numeral 1300, will now be described.

Initially, at step 1302, a full or broad spectrum of the user or patient is acquired via probe 1102. As mentioned above, the exemplary system 1100 is designed so as to acquire a full spectrum between 600 nm and 1000 nm. Different resolutions may be used, for example and without limitation, a resolution of 5 nm from 600 nm to 1000 nm, or 81 wavelengths in total.

At step 1304, the acquired spectral signal or data is analyzed or processed. In some embodiments, it may be preferable to directly send or transmit the acquired raw spectral data to remote device 1122 for analysis (for example to minimize the power requirements of wearable digital processor 1112). In other embodiments, the analysis, processing or pre-processing (i.e. averaging of multiple acquisitions or other) of the acquired spectra may be done, at least in part, via digital processor 1112 located on headband 1104 before being transmitted.

As mentioned above, the high spectral resolution provided by system 1100 provides a higher discrimination ability between various chromophores being monitored. These may include, without limitation, extracting concentrations for chromophores like carbon monoxide, cytochrome oxidase, oxyhemoglobin, deoxyhemoglobin, or other hemoglobin types, melanin, etc. Blood volume changes can also be monitored, for example, where monitored concentrations remain relatively constant but a greater or lesser volume of probed molecules travel across the sensor's field of view over time.

The high resolution and the large range of the acquired spectrum allows, for example, spectral unmixing analysis, wherein the spectral signature can be broken down into its constituent spectral components and the relative proportion of each of these component spectra can be deduced. This has the unique capability of being able to extract known absorption spectra from the at-sensor spectra and find “residual” signatures with spectra of unknown origin. Conversely, a spectral signature can be extracted based only on its unique feature distribution over the entire IR range. This allows better estimates of the material causing that signature. It also allows extraction of spectra with very broad features more accurately, which is not readily available using only a few token wavelengths in a conventional oximeter, since these broad spectra features are more affected by confounding factors. Thus, each spectral component can be resolved or identified. In combination with an analytical model, this allows the calculation of absolute values. This is in contrast with current cerebral oximetry techniques which rely on the calculation of indices (regional saturation, etc.) based on ratios and these can only be relative to baseline measurements. This means that values from one patient to another may vary significantly and comparisons are therefore not easily done.

Different functions or functional forms may be used or fitted to the spectral data to extract distinct chromophore signatures therein. This may include different multivariate analysis methods known in the art for addressing the presence of two or more chromophore components having overlapping spectral features.

Moreover, while conventional cerebral oximeters tend to average readings or measurements over a period of several seconds to be able to output a steady reading, in contrast, system 1100 may be operable, in some embodiments, to deconvolve the physiological parameters for each spectral reading acquired at a high frequency in order to remove any confounding effect and thus be able to render a high-frequency reading of all parameters, which avoids the need to overly average readings to remove those confounding effects.

The nature of spectroximetry, or hyperspectral oximetry, as in the case of system 1100, allows measurement of absolute transmission of energy. Therefore, intra-patient and/or inter-patient measurements can be readily taken, for example, without significant pre-calibration efforts or techniques.

Once one or more chromophore absorption levels have been extracted from the spectral data, in step 1306, these may be correlated with one or more physiological or health-related parameters, or with a change thereof. In some embodiments, the higher level of spectral information acquired by system 1100 may allow to derive correlations with known clinical data using one or more optimization algorithms, for example using AI models or similar (including neural network models or deep-learning models).

Moreover, since the acquired spectral data covers a large band of wavelengths, this allows not only to compare spectra between users or patients, but it also allows customization of the diagnostic value or device response to the target individual.

These one or more physiological or health-related parameters may include, without limitation, blood pressure, blood or tissue oxygenation, pulse, blood flow rate, blood loss or hemorrhaging, cognitive assessments, lung efficiency, rate of O2 consumption by the brain or other physiological system being probed, stress detection, blackout warnings, CPR monitoring, assessment of vital signs, detection of strokes, etc. Some of these will be discussed further below. Moreover, since system 1100 is operable to acquire high-frequency spectral data which contains the presence of multiple chromophore signatures simultaneously, it may thus allow for a perfect synchronization of correlations between the one or more physiological or health-related parameters derived therefrom.

In some embodiments, the absolute nature of the absorption spectra acquired by probe 1102 may allow to detect blood loss, or hemorrhaging. For example, while the SpO2 parameter measured using traditional oximetry techniques only considers the fraction of the hemoglobin molecules in the oxygenated state and not the total hemoglobin content, it cannot provide an absolute reference value from one individual to another. Indeed, it can only provide a measure of the portion of hemoglobin molecules which are/are not oxygenated. In contrast, system 1100 is operable to provide a more complete spectrum and may thus be able to assess the level of hemoglobin concentration from the total absorption of light. In the exemplary case of blood loss, system 1100 may detect the total concentration of hemoglobin going down, even when the oxygen saturation remains at 100%.

In some embodiments, system 1100 may provide diagnostic evaluation via the use of bolus type tests in which an “indicator” (e.g. naturally occurring or foreign tracer molecule or similar) is introduced in the blood and its effects are measured. For example, a patient may receive a shot of high concentration O₂, which may be detected via a spike in measured venous oxyhemoglobin, which may be detected in the head or other monitored region. This type of measurement would not be possible with conventional pulse oximetry methods or systems. If the initial amount of O₂ introduced is known, system 100 may derive therefrom a concentration of new oxyhemoglobin, which, combined with a measurement of the change in absorption of light in the head (or other region), may be used to derive a venous blood optical “thickness” value. The same process may also be done when monitoring arterial oxyhemoglobin and consequently a corresponding proportion of arterial to venous content in the head can be derived. For instance, if the amount of new arterial oxyhemoglobin resulting from the “shot” is estimated, then changes measured in the oxyhemoglobin absorption can be fitted to a venous volume required to manifest the total spectral absorption observed, thereby providing an indication as to arterial to venous proportions. Other tests may include, but are not limited to, pulmonary efficiency, in that knowing an increase in O₂ molecules introduced, one can measure what amount reaching the blood (e.g. via spectral absorption) and qualify or quantify a proportion of the O₂ being absorbed into the blood and a speed or efficiency at which it does. These and other similar tests may be done by system 1100 in real-time for each individual.

In some embodiments, the same “bolus” type test may also be used to provide the time of travel between the lungs and a point of interest on the body (e.g. head; extremities such as arms, fingers, feet or legs as a function of blood pressure). This type of measurement may be used to derive a blood flow rate value, for example.

Similarly, since blood flow rate is dependent on blood pressure, similar correlations between flow rates and blood pressure may be derived. Currently, correlations derived using a conventional pulse-oximetry signal and blood pressure are statistically-based and use databases of previously measured signals to optimize an algorithm (such as AI). In contrast, the full Spectrum approach provided by system 100 is more versatile as it is based on direct correlations between different physiological parameters.

In some embodiments, detected changes in oxyhemoglobin and deoxyhemoglobin may be combined with the O₂ content being breathed (e.g. the % O₂ being breathed), to derive a level of dissolved oxygen in the blood.

In some embodiments, system 1100 may be configured to detect an increase in the optical density related to oxyhemoglobin in the venous blood, and may use the concentration of O₂ being breathed (e.g. the % O₂ being breathed), to derive a corresponding hemoglobin concentration. This may also be done when measuring a decrease in optical density of oxyhemoglobin with a decreasing concentration of O₂ being breathed.

In some embodiments, system 1100 may be used to monitor O₂ delivery. For example, in some clinical settings, it may be desirable to administer O₂ to a patient to increase the partial pressure of O₂ in the patient's lungs and the blood. However, elevated concentrations of O₂ in the blood for prolonged time are known to have detrimental effects. Conventional pulse oximeters are not able to show if the patient is in a hyperoxic state (or above partial pressure of 0.21 ATA). In contrast, full spectrum oximetry as provided by system 1100 may be operable to track elevated O₂ states. It may also be operable to detect dropping O₂ levels before a hypoxic state is even reached, in contrast to a pulse oximeter that would typically only be able to detect the hypoxic state once reached.

In some embodiments, system 1100 may be used in hyperbaric medicine. For example, in some embodiments, system 100 may be configured to track hyperoxic states well above a O₂ partial pressure of 0.21 ATA, thus allowing the monitoring of how close the patient is to hazardous levels of oxygen toxicity.

In some embodiments, system 1100 may be used for cognitive assessment during sports or in extreme environments. For example, system 1100 may be configured to provide assessment of oxygenation levels during exercise. It is well known that conventional oximetry does not see or detect increases in blood oxygenation beyond SpO2 of 100%, which is very close to the value anyone has normally at rest. In contrast, system 1100 may be operable to see or detect increases in the level of oxyhemoglobin reaching the organ of interest (e.g. brain) in a specified unit of time during exercise. For instance, this may be used to indicate an increase in blood flow, and thus of oxyhemoglobin, to the organ under observation, which translates in a greater delivery of O₂ to that organ. Thus, in some embodiments, system 1100 may be used to monitor or assess the level of increased oxygenation from one activity to another, which may be used to create a baseline by finding normal increases in oxygen delivery to the brain (or other organs) using a sample population. Thus, measurements from an individual may be compared to this baseline and this used to assess performance, impairments, etc.

In some embodiments, system 1100 may be configured to monitor the rate of O₂ consumption in the brain or other organ of interest. For example, with normal air, arterial blood is almost 100% saturated. If 100% O₂ is breathed, the venous deoxyhemoglobin in the organ will decrease by an amount proportional to the amount of 0₂ not metabolized by the tissue under study. Thus, by knowing the input quantity or amount of air and knowing what is left over from the dissolved O₂ that went back into the venous hemoglobin (thus raising its oxyhemoglobin content), system 1100 may be configured to derive the portion of O₂ taken up by the organ of interest. In some embodiments, this may be done on a population sample which may then be used as a reference or baseline for diagnostic of other individuals.

In some embodiments, system 1100 may be operable to derive a lung efficiency value or similar.

By introducing a known increase in O2 content being breathed and measuring the effective change in oxy and deoxy hemoglobin, and if applicable knowing the metabolized amount in the tissue under investigation, system 100 may derive therefrom a measure of efficiency of O2 transfer occurring at the pulmonary level. Conversely, O₂ intake may be reduced and system 100 used to monitor the corresponding decrease of oxyhemoglobin optical density in the arteries.

In some embodiments, system 1100 may be operable to derive a concentration of O₂ breathed. This may be done as described above for assessing lung efficiency, but here assuming a fixed level of lung efficiency to derive the concentration being breathed. In some embodiments, system 1100 may thus be combined or used in conjunction with a rebreather diving apparatus or similar.

In some embodiments, system 1100 may be used for stress detection and assessment. For example, stress in a user or patient impacts physiological parameters such as pulse, respiration, and blood flow rate. All these parameters may be correlated to the spectra recorded via system 1100. An assessment on the stress level can be made using a combination of known states for each parameter as well as known changes to these parameters (i.e. sudden increase in heart rate and breathing).

In some embodiments, system 1100 may be configured to alert for imminent blackout in an individual or user. The onset of blackout in a user may be predicted based on the oxygenation state of the person. For example, for military pilots, a drop of blood flow to the brain, or a drop in oxygenation levels may be used to mitigate risk of blackouts.

In some embodiments, system 1100 may be used to monitor cardiopulmonary resuscitation (CPR) maneuvers or the like. Currently, CPR is performed using set recommended protocols and procedures for the frequency of chest compressions and mouth-to-mouth assisted breathing. These protocols are established based on experience. Means for an assessment of the performance of CPR given to a patient in real-time while CPR is administered can significantly improve patient outcome. The protocol could be adapted to the patient's needs given specific scenario and response. However, one of the problems with traditional cerebral oximetry is the lack of common baseline from one patient to another. It is also not clear what values given by one instrument should be used as target since (1) the index is relative, (2) indices are not calculated the same way, (3) the same index can vary from one device to the next due to design factors, (4) lack of clinical studies across all devices, (5) variability in readings from one patient to another using the same device given skin type, ethnic background, etc. Another significant disadvantage of traditional cerebral oximetry is the fact that the index typically is calculated using an integration time significantly longer than a normal heartbeat. Sensitivity to minute changes in hemoglobin is therefore compromised. Finally, conventional pulse oximetry will not work when the patient has weak or no pulse.

Full spectrum oximetry as provided by system 1100 may allow for the instantaneous assessment of vital signs. For example, by deconvolving spectral signatures, individual contribution of each type of chromophore may be measured. High-frequency measurements can see variations in blood flow that could be indicative of chest compressions. This approach can also define a target “absolute index” of absorption in the brain caused by oxyhemoglobin, blood flow, and other useful parameters. This index can be then be used for all individuals.

In some embodiments, system 1100 may be configured to detect strokes resulting from the blockage of blood flow to the brain. As discussed above, reduction of blood flow may be derived by system 1100 via a significant reduction in absorption of key spectral indicators.

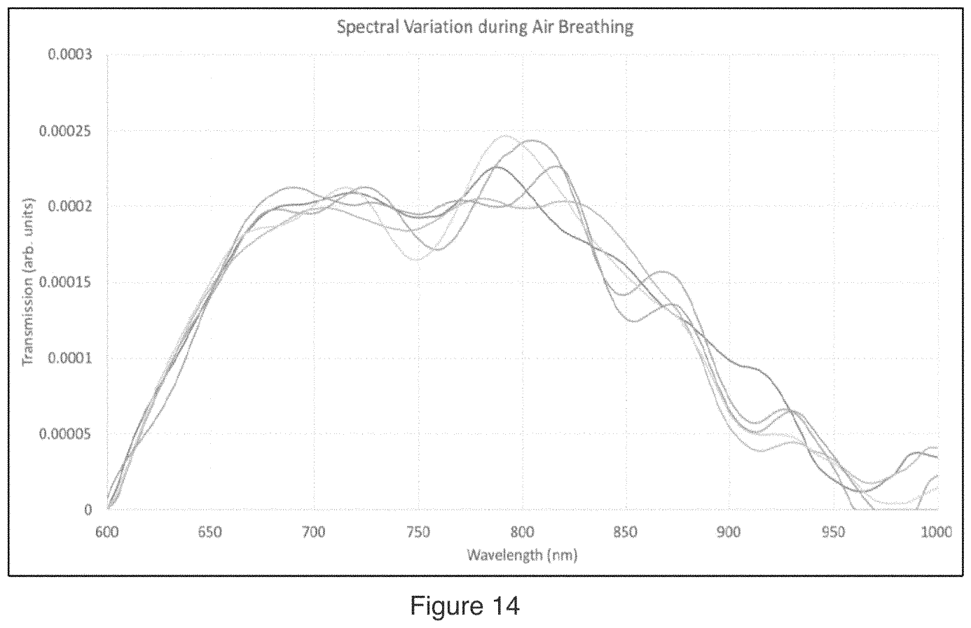

With reference to FIGS. 14 to 16, and in accordance with one exemplary embodiment, an exemplary set of measurements acquired using an exemplary embodiment of system 1100 will be discussed.

FIG. 14 shows an exemplary plot of multiple spectral transmission curves acquired at the cerebral level for a user wearing system 1100 breathing normal air while being in a seated position. The plot shows randomly acquired spectra over a two-minute period. The time sampling of repeated measurements (i.e. different curves) shows variations that are due to the inherent physiological changes caused by varying blood flow (heart beats, blood pressure, etc.), breathing rate, and other such normal body functions. Spectra taken at various times therefore will show variations in the acquired spectra due to these inherent physiological changes. This allows to derive values for physiological parameters such as pulse, blood flow, head orientation, blood volume, blood pressure, etc. with adequate modeling, since the spectral differences can be used to derive the physiological parameters that affect these readings such as pulse, blood volume, blood volume, blood pressure, etc.

In some embodiments, the transmission curve of normal air at 21% (FIG. 14) can represent a baseline to which changes arising from different O2 concentration can be interpreted.

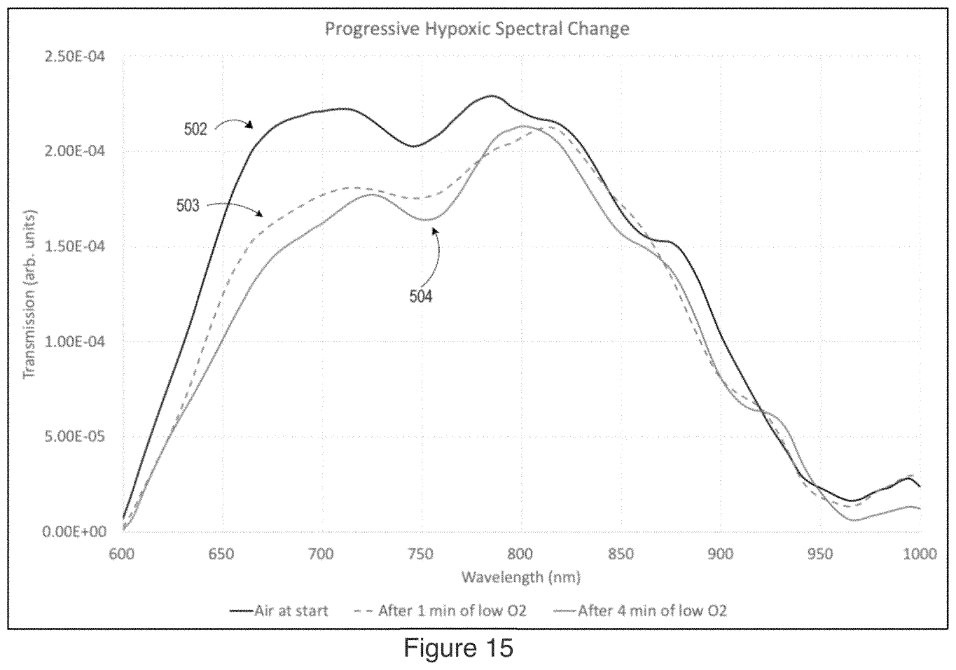

With reference to FIG. 15, a plot showing an average change of spectral readings when switching from normal air breathing, as seen in the previous FIG. 14, to breathing a hypoxic gas that contains 5% O₂ will now be discussed. Breathing a hypoxic gas results in a significant lowering of the level of oxygen reaching the blood and tissues. The plot of FIG. 15 shows the progressive change in spectral readings with line 502 representing an average of multiple spectra taken during one-minute breathing air in a sitting position as in the previous plot of FIG. 14. Meanwhile, line 503 shows the average of multiple spectra taken after a minute of breathing the hypoxic mix. Finally, line 504 is the average of multiple spectra taken after breathing 4 minutes of the hypoxic mix. In this case, there is a clear decrease in the transmission in the 700 nm area which is consistent with an increase in absorption due to elevated deoxyhemoglobin levels.

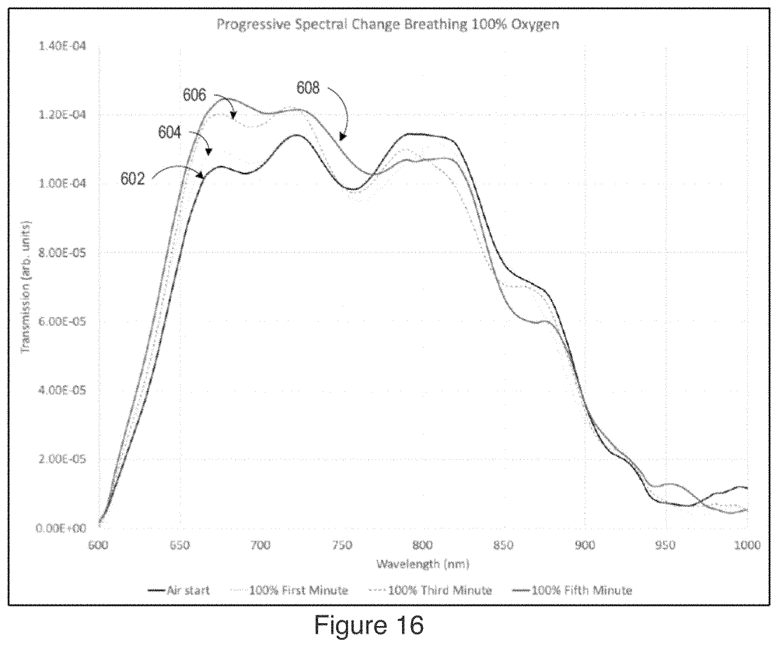

Similarly, FIG. 16 is a plot showing various acquired spectra when the user switches from breathing normal air (21%) to breathing pure oxygen (100%). Line 602 shows the average of multiple spectra taken during the first minute of breathing normal air. Lines 604 and 606 are the average of multiple spectra acquired during the first and third minutes, respectively. Line 608 is the average of multiple spectra acquired during the fifth minute of breathing pure oxygen. While conventional pulse oximeters, as well as cerebral oximeters would not show significant change in these conditions, the full-spectrometric signals acquired via system 1100 clearly show the progressive change related to the changing breathing conditions.

In light of the above, and accordance with one embodiment, a device for real-time vital sign monitoring for individuals in extreme and/or harsh environments is provided, whereby such environments expose users to anomalous environmental respiratory conditions, as described above. Namely, within this context, extreme environments may include any situation where an individual is experiencing lower or higher ambient pressures or requires delivery of oxygen from an external source. For example, these situations include high altitude pilots and underwater divers.

In one such embodiment, the device can detect and track multiple vital signs including heart rate, breathing rate, temperature, and oxygenation. The device consists of a custom light source and a series of optical detectors that operate in a wide range of frequencies, as further detailed below, for example. Light reaching the detectors at specific frequency ranges is tracked and used to derive the physiological parameters of interest, namely those representative of the user's current and/or cumulative physiological response to exposure to the anomalous environmental respiratory conditions. As further detailed below, in this embodiment, operation of the device's optical physiological sensing and monitoring system differs from conventional oximetry in that the device is not dependent on the detection of a pulse to acquire meaningful data.

In one particular embodiment, measurements are made directly at the cerebral level. Consequently, the device is designed to be worn on the forehead where it can be integrated into existing equipment (mask, hoodie, HUD unit, etc.) or as a standalone patch. The device is not limited to this placement and can be also put elsewhere on the body. Outside the scope of underwater applications, the device can also be used for the monitoring of pilot cognitive performance, for example. The device can generally be used in any scenario requiring real-time continuous physiological monitoring.

As introduced above, the device is operable to track oxygen levels in both hypoxic as well as hyperoxic conditions. For this reason, the device is amenable for underwater environments where divers are exposed to significant dangers due to oxygen toxicity.

There are generally no current devices that can track higher than normal oxygenation levels in a non-invasive and continuous manner. Conventional measurement of oxygenation consists of detecting arterial saturation (SpO₂) using a pulse oximeter placed on the extremities of the body (fingers, toes, ear lobes, etc.). In the context of diving, SpO₂ is not very useful as it is only indicative of arterial oxyhemoglobin levels, can only be used for hypoxia, and is ineffective in cases of reduced blood circulation such as in cold temperatures.

Comparatively, devices as described herein in accordance with some embodiments, are operable to detect and track hyperoxia resulting from O₂ partial pressures well above 0.21 ATA as experienced in diving applications.

Accordingly, the device represents a complete vital sign monitoring solution and allows for a continuous adaptation of a dive profile, for example, to the actual physiological condition of the diver in open, semi-closed, and closed circuit scenarios, while also mitigating the dangers of hyperoxia.

To demonstrate the tracking of oxygen during hyperoxia, the herein-described approaches were tested in real underwater environments and in hyperbaric chambers. As an example, FIG. 8 (described above) shows data acquired during a dive in a hyperbaric chamber. The diver is initially at the surface breathing air. A first descent reaches 30 feet (t=600 sec.). After a few minutes, the diver switches to 100% O2 while the depth is kept unchanged. The diver is then brought up to 15 feet where a switch back to air occurs after a few minutes. For reference, the breathing gas and partial pressure (pO2) at each depth is indicated on the graph. It can be readily observed that the light transmission detected by the device correlates directly to the changes in oxygen experienced by the diver.

Two features can be highlighted in FIG. 8. First is the fact that the device follows the changes in oxygen levels actually experienced by the diver at a physiological level and not through any derivation based on depth and gas mix. For example, when the first switch from air to 100% occurs, the transmission levels are instantly reduced (indicating increased oxygen) while the atmospheric pressure remains that of 30 feet depth.

Secondly, the device provides a means to track and mitigate long term exposure to O₂. This is demonstrated by comparing the first period in which the diver is breathing air at 30 feet pressure and the last period where the diver is breathing air at 15 feet pressure. Based only on depth and gas mix, it would be expected that the level of oxygen exposure in the diver's system is less in the latter period at 15 feet than in the initial period at 30 feet. The acquired data however shows slightly increased levels of O₂ exposure at 15 feet. The cause of this is explained by residual O₂ in the diver's system resulting from high exposure while breathing 100% O₂. To our knowledge this figure is the first such recorded evidence demonstrating that partial pressure based on gas mix and depth alone is not sufficient to assess a diver's true exposure to oxygen and risks of oxygen toxicity.

Using this approach, the device is capable of monitoring all vital signs in real-time during a dive and allow for a continuous adaptation of the dive profile to the actual physiological condition of the diver. For example, the diver's real-time physiological data can feedback into the rebreather or dive computer to continuously adapt the dive to the actual physiological state of the diver, independently of the sensors onboard the rebreather.

Tests were conducted to demonstrate the impact of the level of physical activity on oxygen exposure and its associated risks in hyperoxic conditions. For example, FIG. 9 (described above) shows the synchronous tracking of oxygenation, heart rate, and breathing rate as the diver increases the level of physical exertion. The diver descended to 57 feet and performed intense physical activity without altering depth or breathing gas. Shortly after the onset of the physical activity, both the heart rate (middle curve) and the breathing rate (bottom curve) increased sharply. This led to a detectable increase in oxygenation shown by the decrease in light transmission (to curve). The increase in oxygenation is steady and continuous throughout the period of intense activity.

This data suggests the importance of accounting for physiological parameters in the assessment of oxygen exposure and risks of oxygen toxicity.

Accordingly, a computational process as described herein that can take physiological parameters into account can compensate for the variability of a diver's reaction to the same dive conditions at different times. Actionable decisions can then be tailored to the diver's specific condition at a given time. Actionable decisions can include but are not limited to: surface, reduce depth, or allow increased depth; prolong or reduce dive time, alter gas mix; reduce physical activity; etc.

This device allows the significant improvement adding physiological parameters to the current approaches for dive management that are otherwise solely based on environmental conditions (pressure, running time, etc.). Whereas existing algorithms make assumptions based on theoretical models of the physiological reactions to environmental conditions, this approach allows new algorithms that take into account the actual physiological parameters as they are being measured in real-time while the diver is exposed to environmental stresses.

Several advantages to real-time physiology-adapted dive management may thus include, but are not limited to, any combination of: Enhanced prediction of hazardous health states; Optimal physical performance; Ability to train Machine-Learning algorithms (Artificial Intelligence) to tailor predictions to individual divers; Unique customized adapted profiles based on day-to-day conditions; Tracking of performance enhancement over time; Detection of anomalies based on history of physiological response; Objective comparison between diver performances; Etc.

While the present disclosure describes various embodiments for illustrative purposes, such description is not intended to be limited to such embodiments. On the contrary, the applicant's teachings described and illustrated herein encompass various alternatives, modifications, and equivalents, without departing from the embodiments, the general scope of which is defined in the appended claims. Except to the extent necessary or inherent in the processes themselves, no particular order to steps or stages of methods or processes described in this disclosure is intended or implied. In many cases the order of process steps may be varied without changing the purpose, effect, or import of the methods described.

Information as herein shown and described in detail is fully capable of attaining the above-described object of the present disclosure, the presently preferred embodiment of the present disclosure, and is, thus, representative of the subject matter which is broadly contemplated by the present disclosure. The scope of the present disclosure fully encompasses other embodiments which may become apparent to those skilled in the art, and is to be limited, accordingly, by nothing other than the appended claims, wherein any reference to an element being made in the singular is not intended to mean “one and only one” unless explicitly so stated, but rather “one or more.” All structural and functional equivalents to the elements of the above-described preferred embodiment and additional embodiments as regarded by those of ordinary skill in the art are hereby expressly incorporated by reference and are intended to be encompassed by the present claims. Moreover, no requirement exists for a system or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, as may be apparent to those of ordinary skill in the art, are also encompassed by the disclosure.

What is claimed is:

1. A patient health monitoring system comprising:
an epidermal device to be affixed to the patient's skin and comprising:
an optical spectroscopy probe operable to acquire data representative of blood oxygenation levels; and
a wireless interface;
a mobile application operable on a mobile device to interface with said wireless interface and receive therefrom said acquired data;
stored computer-executable code operable by a digital processor to monitor for variations in said blood oxygenation levels and digitally evaluate said variations against preset variations corresponding to benchmark blood oxygenation profiles, wherein said profiles are digitally associated with a preset blood oxygenation index defining at least a lower health risk rating and a higher health risk rating, and output a signal representative of said higher health risk rating in response to said evaluation.

2. The system of claim 1, wherein said blood oxygenation levels comprise deoxyhemoglobin concentrations, and wherein said benchmark blood oxygenation profiles comprises deoxyhemoglobin concentration profiles.

3. The system of claim 1, wherein said blood oxygenation levels comprise respective deoxyhemoglobin concentrations and oxidized hemoglobin concentrations, and wherein said benchmark blood oxygenation profiles comprise dissolved oxygen profiles derived from said concentrations.

4. The system of claim 1, wherein said stored computer-executable code is stored on said mobile device and operable by said mobile application, or is stored on a server communicatively accessible by said mobile application via said mobile device.

5. The system of claim 1, further comprising a remote server operatively linked to said mobile application to process acquired data from multiple users in continuously or periodically optimizing said benchmark profiles accordingly.

6. The system of claim 1, wherein said epidermal device comprises an integrated epidermal patch further comprising a body temperature sensor.

7. The system of claim 1, wherein said epidermal device comprises a cerebral device to be affixed to the user's head.

8. The system of claim 1, wherein said optical spectroscopy probe comprises a broad-spectrum oximetry probe comprising: a broad-spectrum light source providing broad-spectrum illumination in probing multiple blood-related chromophores exhibiting distinguishable spectral responses; and a spectrometer operable to acquire an optical signal resulting from said broad-spectrum illumination so to digitally capture said distinguishable spectral responses, wherein said blood-related chromophores are representative of said blood oxygenation levels; wherein said stored computer-executable code is operable by said digital processor to spectrally resolve said distinguishable spectral responses from said optical signal to isolate a spectral signature for a designated chromophore; and compare said isolated spectral signature with a designated set of corresponding signatures associated with a discriminable health-related condition; and output said signal representative of said higher health risk rating in response to said evaluation of said health-related indicator representative of said discriminable health-related condition.

9. The system of claim 8, wherein said digital processor is operable to extract an absolute concentration for said designated chromophore from said spectral signature.

10. The system of claim 8, wherein said broad-spectrum light source emits light in a range of about 600 nm to about 1000 nm.

11. The system of claim 8, wherein said spectrometer is operable to isolate respective spectral responses within at least 10 spectral regions within said broad-spectrum illumination.

12. The system of claim 8, wherein said chromophores comprise at least three of carbon monoxide, melanin, cytochrome oxidase, oxyhemoglobin, or deoxyhemoglobin.

13. The system of claim 8, wherein said discriminable health-related condition comprises at least one of: blood or tissue oxygenation, pulse, blood pressure, blood flow rate, blood loss or hemorrhaging, onset of blackouts or change in cognition, lung efficiency, rate of oxygen consumption by an organ of interest, psychological or physiological stress, presence of stroke, or a change in vital signs.

14. The system of claim 8, wherein said digital processor is operable to isolate a combined spectral signature for a designated combination of chromophores; and compare said isolated combined spectral signature with a designated set of corresponding combined signatures associated with said discriminable health-related condition.

15. A geographical health monitoring system comprising:
a centralized health-monitoring server;
a set of wearable health-monitoring devices to be affixed to respective users within a geographical area to:
acquire health-related data from each of said respective users over time;
concurrently track a location of each of said respective users; and
communicate information related to said health-related data and said location to said centralized health-monitoring server for tracking;
wherein, for each of said respective users, said health-related data is digitally compared with a designated health-related profile associated with a designated medical condition to automatically output a health risk indicator for a given location upon given health-related data acquired at said given location substantially aligning with said designated health-related profile.

16. The system of claim 15, wherein a geographical outbreak is automatically identified upon a group of said health risk indicators are output for a given area around a same said given location.

17. The system of claim 15, wherein infection transmissions are automatically tracked by tracking a geographical evolution of said health risk indicators over time.

18. The system of claim 17, wherein geographical tracking of asymptomatic users is automatically implemented and retroactively evaluated by a digital processor upon any of said asymptomatic users later triggering a said health risk indicator so to track potential retroactive geographical infection transmission from said asymptomatic users.

19. The system of claim 15, wherein said designated health-related profile comprises a combination of at least two of a designated body temperature threshold, a blood oxygen-concentration related threshold or profile, a respiration rate or variation profile, a cardiac rate or variation profile, or a blood pressure or variation profile.

20. The system of claim 15, wherein each of said a set of wearable health-monitoring devices comprises an epidermal device to be affixed to the patient's skin and comprising: an optical spectroscopy probe operable to acquire data representative of blood oxygenation levels; wherein the system further comprises stored computer-executable code operable by a digital processor to monitor for variations in said blood oxygenation levels and digitally evaluate said variations against preset variations corresponding to benchmark blood oxygenation profiles, wherein said profiles are digitally associated with a preset blood oxygenation index defining at least a lower health risk rating and a higher health risk rating associated with said designated medical condition.

21. The system of claim 20, wherein said optical spectroscopy probe comprises a broad-spectrum oximetry probe comprising: a broad-spectrum light source providing broad-spectrum illumination in probing multiple blood-related chromophores exhibiting distinguishable spectral responses; and a spectrometer operable to acquire an optical signal resulting from said broad-spectrum illumination so to digitally capture said distinguishable spectral responses, wherein said blood-related chromophores are representative of said blood oxygenation levels; wherein said stored computer-executable code is operable by said digital processor to spectrally resolve said distinguishable spectral responses from said optical signal to isolate a spectral signature for a designated chromophore; and compare said isolated spectral signature with a designated set of corresponding signatures associated with said designated medical condition.
